(19) United States
(12) Patent Application Publication
(10) Pub. No.: US 2022/0046994 A1
(43) Pub. Date: Feb. 17, 2022

(54) VAPORIZATION DEVICE WITH VAPOR COOLING

(71) Applicant: HEXO OPERATIONS INC., Ottawa (CA)

(72) Inventors: Patrick WOODS, Ottawa (CA); Max ALSAYAR, Ottawa (CA); Guy DEGRACE, Ottawa (CA)

(21) Appl. No.: 17/416,715

(22) PCT Filed: Dec. 20, 2019
(86) PCT No.: PCT/CA2019/051879

§ 371 Date: Jun. 21, 2021

Related U.S. Application Data
(60) Provisional application No. 62/783,369, filed on Dec. 21, 2018.
(60) Provisional application No. 62/792,599, filed on Jan. 15, 2019.
(60) Provisional application No. 62/938,996, filed on Nov. 22, 2019.

(51) Int. Cl. A24F 40/485; A24F 40/51; A24F 40/57; A24F 40/60

(52) U.S. Cl. CPC A24F 40/485; A24F 40/60; A24F 40/57; A24F 40/51

(57) ABSTRACT

A vaporization apparatus includes an atomizer, a channel, and a cooler. The atomizer is provided to generate vapor from a vaporization substance by heating the vaporization substance, and the channel is in fluid communication with the atomizer, to enable fluid flow through the vaporization apparatus. The cooler is provided to cool the fluid.

### CROSS-REFERENCE TO RELATED APPLICATIONS

This application is related to, and claims priority to, U.S. Provisional Patent Application No. 62/783,369, entitled “APPARATUS AND METHODS FOR SERIAL CONFIGURATIONS OF MULTI-CHAMBER VAPORIZATION DEVICES”, and filed on Dec. 21, 2018; U.S. Provisional Patent Application No. 62/792,599, entitled “VAPORIZATION DEVICE WITH RESIDUE PREVENTION OR REDUCTION”, and filed on Jan. 15, 2019; and U.S. Provisional Patent Application No. 62/938,996, entitled “VAPORIZATION DEVICE WITH VAPOR COOLING”, and filed on Nov. 22, 2019, the entire contents of each of which are incorporated by reference herein.

### FIELD

This application relates generally to vaporization devices, and in particular to vaporization devices with features to manage vapor temperature.

### BACKGROUND

A vaporization device is used to vaporize substances for inhalation. These substances are referred to herein as vaporization substances, and could include, for example, cannabis products, tobacco products, herbs, and/or flavorants. In some cases, substances in cannabis, tobacco, or other plants or materials extracted to generate concentrates are used as vaporization substances. These substances could include cannabinoids from cannabis, and nicotine from tobacco. In other cases, synthetic substances are artificially manufactured. Terpenes are common flavorant vaporization substances, and could be generated from natural essential oils or artificially.

Vaporization substances could be in the form of loose leaf in the case of cannabis, tobacco, and herbs, for example, or in the form of concentrates or derivative products such as liquids, waxes, or gels, for example. Vaporization substances, whether intended for flavor or some other effect, could be mixed with other compounds such as propylene glycol, glycerin, medium chain triglyceride (MCT) oil and/or water to adjust the viscosity of a final vaporization substance.

In a vaporization device, the vaporization substance is heated to the vaporization temperature of one or more constituents of the vaporization substance. This produces a vapor, which may also be referred to as an aerosol. The vapor is then inhaled by a user through a channel that is provided in the vaporization device, and often through a hose or pipe that is part of or attached to the vaporization device.

Typical vaporization substances have vaporization temperatures that exceed 100° C. Some users may experience discomfort and irritation when inhaling vapor at these temperatures.

### SUMMARY

According to an aspect of the present disclosure, a vaporization apparatus includes: an atomizer to generate vapor from a vaporization substance by heating the vaporization substance; a channel, in fluid communication with the atomizer, to enable fluid flow through the vaporization apparatus; and a cooler to cool the fluid.

The cooler may be thermally coupled to the channel, and is in fluid communication with the channel in some embodiments. At least a portion of the cooler may even be located inside the channel.

The channel may include an air intake channel, in fluid communication with the atomizer, to carry air to the atomizer, in which case the cooler may be thermally coupled to or in fluid communication with the air intake channel. At least a portion of the cooler is located inside the air intake channel.

In some embodiments, the cooler includes a cooling air intake channel, in fluid communication with the channel, to admit cooling air into the channel to mix with the vapor. The vaporization apparatus may include a regulator to control a flow of the cooling air through the cooling air intake channel.

The cooler may be or include a passive cooling element. A passive cooling element may include a thermally conductive material such as copper to transfer heat away from the fluid, for example.

A vaporization apparatus may also or instead include a cooler that includes an active cooling element, such as a thermoelectric cooling element.

In some embodiments, the cooler includes a surface area increasing structure to increase a surface area for heat transfer. Examples of surface area increasing structures include fins and a coil.

The cooler may also or instead include a heat sink. A heat sink may include any one or more of: air, a liquid, a phase change material. The cooler may include a heat exchanger to transfer heat to the heat sink.

The vaporization apparatus may also or instead include a heat exchanger to transfer heat to the atomizer.

In an embodiment in which the vaporization apparatus includes a chamber to store the vaporization substance, the cooler may include a heat exchanger to transfer heat to the chamber.

Another example of a heat exchanger that may be provided in a cooler is a heat exchanger to transfer heat away from the channel.

The cooler may include a removable cooling element. For example, the removable 2 0 cooling element may be coupled to the vaporization apparatus by a releasable coupling. The removable cooling element is coupled to the vaporization apparatus magnetically in some embodiments.

A vaporization apparatus may also include a power source to power the cooler. Such a power source may be further arranged to power other components such as the atomizer.

A sensor to measure a temperature of the fluid is also provided in some embodiments, and a controller may be coupled to the sensor to control the cooler responsive to a temperature measurement by the sensor.

The vaporization apparatus may include a user input device to receive input from a user; and a controller, coupled to the user input device, to control the cooler responsive to the input from the user.

A vaporization apparatus may include other components, such as a mouthpiece to enable inhalation of the vapor by a user through the channel. The mouthpiece may include at least a portion of the cooler. In an embodiment, the cooler includes a further channel, in fluid communication with the channel and the mouthpiece.

A method of use of such a vaporization apparatus may involve initiating vaporization of the vaporization substance to produce the vapor; and inhaling the vapor through the channel. Such a method may also involve initiating cooling of the vapor by the cooler prior to inhaling the vapor.

Another method involves providing an atomizer for a vaporization apparatus to generate vapor from a vaporization substance by heating the vaporization substance; providing a channel to enable fluid flow through the vaporization apparatus; and providing a cooler to cool the fluid.

Providing the cooler may involve: providing, as the cooler, a cooler to be thermally coupled to the channel, a cooler that is to be in fluid communication with the channel, and/or a cooler that is to be at least partially located inside the channel.

The channel may include an air intake channel to carry air to the atomizer, in which case providing the cooler may involve providing, as the cooler, a cooler that is thermally coupled to the air intake channel, a cooler that is to be in fluid communication with the air intake channel, and/or a cooler that is to be at least partially located inside the air intake channel.

Providing the cooler may also or instead involve providing, as the cooler, a cooler that includes a cooling air intake channel to admit cooling air into the channel to mix with the vapor. In some embodiments, a method may also involve providing a regulator to control a flow of the cooling air through the cooling air intake channel.

Providing the cooler may involve providing, as the cooler, a cooler that includes a passive cooling element. The passive cooling element may include a thermally conductive material such as copper, to transfer heat away from the fluid.

A cooler may include an active cooling element, and therefore providing the cooler may involve providing, as the cooler, a cooler that includes an active cooling element. The active cooling element may include a thermoelectric cooling element, for example.

Providing the cooler may also or instead involve providing, as the cooler, a cooler that includes a surface area increasing structure, such as fins and/or a coil, to increase a surface area for heat transfer.

In some embodiments, providing the cooler involves providing, as the cooler, a cooler that includes a heat sink. The heat sink may include any one or more of: air, a liquid, and a phase change material. The cooler may include a heat exchanger to transfer heat to the heat sink.

The cooler may include a heat exchanger to transfer heat to the atomizer.

A method may involve providing a chamber to store the vaporization substance, in which case the cooler may include a heat exchanger to transfer heat to the chamber.

The cooler may also or instead include a heat exchanger to transfer heat away from the channel.

In some embodiments, the cooler includes a removable cooling element. The removable cooling element may be couplable to the vaporization apparatus by a releasable coupling. For example, the removable cooling element may couplable the vaporization apparatus magnetically.

A method may involve providing a power source to power the cooler. Providing the power source may involve providing the power source to further power the atomizer.

Some embodiments involve providing a sensor to measure a temperature of the fluid, and providing a controller to control the cooler responsive to a temperature measurement by the sensor.

A method may also or instead involve providing a user input device to receive input from a user; and providing a controller to control the cooler responsive to the input from the user.

Other components may also or instead be provided. For example, a method may involve providing a mouthpiece to enable inhalation of the vapor by a user through the channel. The mouthpiece may include at least a portion of the cooler, and/or the cooler may include a further channel to be in fluid communication with the channel and the mouthpiece.

Other aspects and features of embodiments of the present disclosure will become apparent to those ordinarily skilled in the art upon review of the following description.

### BRIEF DESCRIPTION OF THE DRAWINGS

For a more complete understanding of the present disclosure, reference is now made to the following description taken in conjunction with the accompanying drawings, in which:

FIG. 1 is a plan view of an example vaporization device;

FIG. 2 is an isometric view of the vaporization device in FIG. 1;

FIG. 3 is an isometric view of another example vaporization device;

FIG. 4 is a diagram illustrating internal structure of an example vaporization device tank with a ceramic core;

FIG. 5 is a block diagram illustrating a cooler according to an embodiment;

FIG. 6 is a block diagram illustrating an example vaporization device with a cooler;

FIG. 7 is an isometric and partially exploded view of another example vaporization device with a cooler;

FIG. 8 is an isometric and partially exploded view of a further example vaporization device, which includes a cooler in the form of a heat sink;

FIG. 9 is an isometric and partially exploded view of yet another example vaporization device with a cooler;

FIG. 10 is a top view of an example chamber with a cooler;

FIG. 11 is a cross-sectional view of the chamber illustrated in FIG. 10, along the line A-A in FIG. 10;

FIG. 12 is a plan and partially exploded view of another example vaporization device with a cooler;

FIG. 13 is a top view of a chamber illustrated in FIG. 12;

FIG. 14 is a cross-sectional view of the chamber illustrated in FIG. 13, along the line B-B in FIG. 13;

FIG. 15 is a cross-sectional view of another example chamber with a cooler located at a position inside a channel;

FIG. 16 is a diagram illustrating internal structure of an example vaporization device cartridge with a cooler;

FIG. 17 is a plan view of an example cap with a cooler;

FIG. 18 is a plan view of another example cartridge that includes longer channels;

FIG. 19 is a block diagram illustrating an example vaporization device with a cooler upstream of an atomizer;

FIG. 20 is a diagram illustrating internal structure of an example vaporization device tank with a cooler in an air intake channel;

FIG. 21 is a plan view of a cap according to another embodiment;

FIG. 22 is a cross-sectional and partially exploded view of an example of engagement structures in a vaporization device;

FIG. 23 is a flow diagram illustrating a method according to an embodiment;

FIG. 24 is a flow diagram illustrating a method according to another embodiment.

### DETAILED DESCRIPTION

The performance of a vaporization device can depend on the temperatures achieved during vaporization. In some cases, vaporizing at high temperatures can have potential advantages. For example, a vaporization substance may be vaporized more rapidly at higher temperatures than at lower temperatures, thereby providing a larger amount or quantity of vapor that is available for a user to inhale. High temperatures can also help ensure that the various constituents of a vaporization substance are vaporized, including any or all cannabinoids, nicotine, and/or terpenes, for example. This could result in a higher quality vapor that provides the user with a fuller effect or experience that the vaporization substance is intended to provide. However, combustion of at least some constituents of a vaporization substance could also occur when vaporizing at high temperatures, which can produce a vapor with an undesirable burnt taste and/or aroma. Therefore, vaporization devices often avoid heating vaporization substances to temperatures that result in combustion. In some embodiments, target temperatures for vaporization are in the range of 150° C. to 180° C.

Vapor temperature can impact user experience, in that inhaling high temperature vapor may cause discomfort and irritation for a user, which can lead to coughing. Therefore, some users may desire or prefer a vaporization device that provides a lower temperature vapor for inhalation. For example, although a normal operating temperature range of a vaporization device may be 100° C. to 250° C., a comfortable temperature of vapor for user inhalation may be below about 150° C., but a preferred maximum temperature for vapor may vary between different users.

Although vaporizing a vaporization substance at higher temperatures may be appealing from a vaporization device performance point of view, user comfort could be diminished at these higher temperatures because of the hotter vapor that is produced. On the other hand, vaporization at lower temperatures may only produce a thin or weak vapor in low quantities. For example, vaporization at lower temperatures could result in incomplete vaporization of a vaporization substance, and produce a vapor that has relatively low concentrations of at least some constituents of the vaporization substance.

Therefore, there is a trade-off between achieving suitable performance and managing user comfort in some vaporization devices. In the case that high temperatures are used to rapidly and thoroughly vaporize a vaporization substance, a relatively high quantity and quality of vapor could be produced. However, the resulting vapor might also have a high temperature and result in discomfort for the user. In the case that low temperatures are used to produce a vapor that is at a comfortable temperature for the user, the quality and/or quantity of the vapor could be reduced.

A need exists for vaporization devices that can not only provide a high quality and quantity of vapor, but also manage vapor temperature to help ensure user comfort. According to some embodiments disclosed herein, vaporization devices include features to cool a vapor 2 0 before inhalation by a user. Vaporization in these devices can occur at relatively high temperatures, and the temperature of the resulting vapor can be reduced using the cooler to provide a more pleasurable experience for the user.

For illustrative purposes, specific example embodiments will be explained in greater detail below in conjunction with the figures. It should be appreciated, however, that the present disclosure provides many applicable concepts that can be embodied in any of a wide variety of contexts. The specific embodiments discussed are merely illustrative and do not limit the scope of the present disclosure. For example, relative to embodiments shown in the drawings and/or referenced herein, other embodiments may include additional, different, and/or fewer features. The figures are also not necessarily drawn to scale.

The present disclosure relates, in part, to vaporization apparatus such as vaporization devices for vaporization substances that include substances such as cannabinoids or nicotine. However, the vaporization devices described herein could also or instead be used for other types of vaporization substances.

As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

For the purpose of this specification, the expression “cannabinoid” means a compound such as tetrahydrocannabinol (THC), cannabidiol (CBD), cannabigerolic acid (CBGA), cannabigerol (CBG), cannabigerol monomethylether (CBGM), cannabigerovarin (CBGV), cannabichromene (CBC), cannabichromevarin (CBCV), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

In some embodiments, the cannabinoid is CBD. For the purpose of this specification, the expressions “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-1-yl)-5-pentyl-1,3-benzenediol); (2) Δ4-cannabidiol (2-(6-isopropenyl-3-methyl-4-cyclohexen-1-yl)-5-pentyl-1,3-benzenediol); (3) Δ3-cannabidiol (2-(6-isopropenyl-3-methyl-3-cyclohexen-1-yl)-5-pentyl-1,3-benzenediol); (4) Δ3-cannabidiol (2-(6-isopropenyl-3-methylenecyclohex-1-yl)-5-pentyl-1,3-benzenediol); (5) Δ2-cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In some embodiments, the cannabinoid is THC. THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A vaporization substance may include a cannabinoid in its pure or isolated form or in a source material that includes the cannabinoid. The following are non-limiting examples of source materials that include cannabinoids: cannabis or hemp plant material (e.g., flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials that include cannabinoids are combined with water, lipids, hydrocarbons (e.g., butane), ethanol, acetone, isopropanol, or mixtures thereof.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some embodiments, the cannabinoid is a mixture of THC and CBD. The w/w ratio of THC to CBD in the vaporization substance may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some embodiments, a vaporization substance may include products of cannabinoid metabolism, including 11-hydroxy-Δ9-tetrahydrocannabinol (11-OH-THC).

These particulars of cannabinoids are intended solely for illustrative purposes. Other embodiments are also contemplated.

As used herein, the term “terpene” (or “decarboxylated terpene”, which is known as a terpenoid) is generally understood to include any organic compound derived, biosynthetically for example, from units of isoprene. Terpenes may be classified in any of various ways, such as by their sizes. For example, suitable terpenes may include monoterpenes, sesquiterpenes, or triterpenes. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thuj one, citronellol, 1,8-cineole, cycloartenol, and derivatives thereof. Further examples of terpenes are discussed in US Patent Application Pub. No. US2016/0250270.

In general, a vaporization substance includes one or more target compounds or components. A target compound or component need not necessarily have a psychoactive effect. One or more flavorants, such as any one or more of: terpene(s), essential oil(s), and volatile plant extract(s), may also or instead be a target compound for vaporization in order to provide flavor to a vapor flow. A vaporization substance may also or instead include other compounds or components, such as one or more carriers. A carrier oil is one example of a carrier.

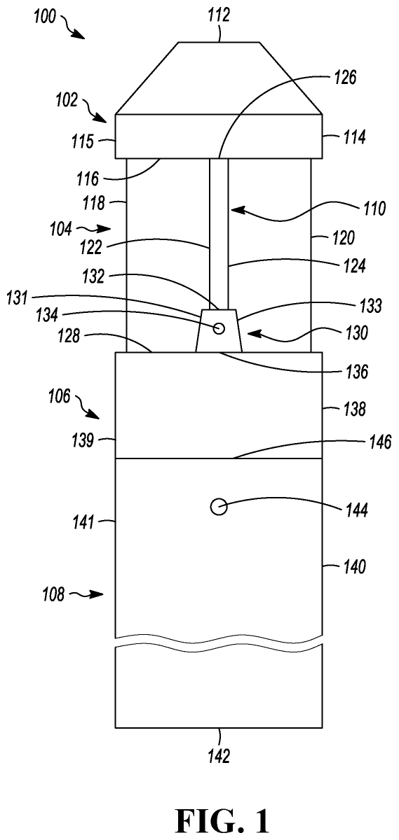

Turning now to vaporization devices in more detail, FIG. 1 is a plan view of an example vaporization device 100. In FIG. 1, the vaporization device 100 is viewed from the side. The vaporization device 100 could also be referred to as a vaporizer, a vaporizer pen, a vape pen or an electronic or “e-” cigarette, for example. The vaporizer 100 includes a cap 102, a chamber 104, a base 106 and a battery compartment 108.

The cap 102 is an example of a lid or cover, and includes a tip 112 and sidewalls 114 and 115, which are sides or parts of the same cylindrical sidewall in some embodiments. The cap 102, in addition to sealing an end of an interior space of the chamber 104, also provides a mouthpiece through which a user can draw vapor from the vaporization device 100 in some embodiments. The mouthpiece is tapered as shown in FIG. 1, and/or otherwise shaped for a user's comfort. The present disclosure is not limited to any particular shape of the cap 102.

The cap 102 could be made from one or more materials including metals, plastics, elastomers and ceramics, for example. However, other materials may also or instead be used.

In other embodiments, a mouthpiece is separate from the cap 102. For example, a cap may be connected to a mouthpiece by a hose or pipe that accommodates flow of vapor from the cap to the mouthpiece. The hose or pipe may be flexible or otherwise permit movement of the mouthpiece relative to the cap, allowing a user to orient the mouthpiece independently from the cap.

The chamber 104 is an example of a vessel to store a vaporization substance prior to vaporization. Although embodiments are described herein primarily in the context of vaporization liquids such as oil concentrates, in general a chamber may store other forms of vaporization substances, including waxes and gels for example. Vaporization substances with water-based carriers are also contemplated. A vaporization device may be capable of vaporizing water-based carriers with emulsified cannabinoids, for example. The chamber 104 may also be referred to as a container, a housing or a tank.

The chamber 104 includes outer walls 118 and 120. Although multiple outer walls are shown in FIGS. 1 at 118 and 120, the chamber 104 is perhaps most often cylindrical, with a single outer wall. The outer walls 118 and 120 of the chamber 104 may be made from one or more transparent or translucent materials, such as tempered glass or plastics, in order to enable a user to visibly determine the quantity of vaporization substance in the chamber. The outer walls 118 and 120 are made from one or more opaque materials such as metal alloys, plastics or ceramics in some embodiments, to protect the vaporization substance from degradation by ultraviolet radiation, for example. The outer walls 118 and 120 of the chamber 104 may include markings to aid the user in determining the quantity of vaporization liquid in the chamber. The chamber 104 may have any of a number of different heights and/or other dimensions, to provide different interior volumes.

The chamber 104 engages the cap 102, and may be coupled to the cap, via an engagement or connection at 116. A gasket or other sealing member may be provided between the chamber 104 and the cap 102 to seal the vaporization substance in the chamber.

Some chambers are “non-recloseable” or “disposable” and cannot be opened after initial filling. Such chambers are permanently sealed once closed, and are not designed to be opened and re-sealed. Others are recloseable chambers in which the engagement at 116, between the cap 102 and the chamber 104, is releasable. For example, in some embodiments the cap 102 is a cover that releasably engages the chamber 104 and seals a vaporization substance in the chamber 104. One example of a releasable engagement disclosed elsewhere herein is a threaded engagement or other type of connection, with an abutment between the chamber 104 and the cap 102 but without necessarily an actual connection between the chamber and the cap. Such a releasable engagement permits the cap 102 to be disengaged or removed from the chamber 104 so that the chamber can be cleaned, emptied, and/or filled with a vaporization substance, for example. The cap 102 is then re-engaged with the chamber 104 to seal the vaporization substance inside the chamber.

FIG. 1 also illustrates a stem 110 inside the chamber 104. The stem 110 is a hollow tube or channel through which vapor can be drawn into and through cap 102. The stem 110 may also be referred to as a central column, a central post, a chimney, a hose or a pipe. The stem 110 includes outer walls 122 and 124, although in many embodiments the stem is cylindrical, with a single outer wall. Materials such as stainless steel, other metal alloys, plastics and ceramics may be used for stems such as the stem 110. The stem 110 couples the cap 102 via an engagement or connection 126. Similar to the engagement or connection 116, the engagement or connection 126 is a releasable engagement or connection in some embodiments, and includes a releasable engagement between the stem 110 and the cap 102. In some embodiments, the engagement 126 is in the form of, or includes, a releasable connection.

Although labeled separately in FIG. 1, the engagements at 116 and 126 are operationally related in some embodiments. For example, in some embodiments screwing the cap 102 onto the stem 110 also engages the cap with the chamber 104, or similarly screwing the cap onto the chamber also engages the cap with the stem. This is one example of a threaded connection that also releasably maintains an abutment between the chamber 104 and the cap 102 but without an actual connection between the chamber and the cap.

An atomizer 130 is provided at the base of the stem 110, inside the chamber 104. The atomizer 130 may also be referred to as a heating element, a core, or a ceramic core. The atomizer 130 includes sidewalls 131 and 133, which actually form a single cylindrical or frustoconical wall in some embodiments, and one or more wicking holes or intake holes, one of which is shown at 134. The sidewalls of the atomizer 130 may be made from a metal alloy such as stainless steel, for example. The sidewalls 131 and 133 of the atomizer 130 are made from the same material as the stem 110 in some embodiments, or from different materials in other embodiments.

The atomizer 130 engages, and may couple with, the stem 110 via an engagement 132, and with the base 106 via an engagement 136. Although the engagements 132 and 136 may be releasable, the stem 110, the atomizer 130, and the base 106 are permanently attached together in some embodiments. The atomizer sidewalls 131 and 133 may even be formed with the stem 110 as an integrated single physical component.

In general, the atomizer 130 converts the vaporization substance in the chamber 104 into a vapor, which a user draws from the vaporization device 100 through the stem 110 and the cap 102. Vaporization liquid is drawn into the atomizer 130 through the wicking hole 134 and a wick in some embodiments. The atomizer 130 may include a heating element, such as a resistance coil around a ceramic wick, to perform the conversion of vaporization liquid into vapor. A ceramic atomizer may have an integrated heating element such as a coiled wire inside the ceramic, similar to rebar in concrete, in addition to or instead of being wrapped in a coiled wire. A quartz heater is another type of heater that may be used in an atomizer.

In some embodiments, the combination of the atomizer 130 and the chamber 104 is referred to as a cartomizer.

The base 106 supplies power to the atomizer 130, and may also be referred to as an atomizer base. The base 106 includes sidewalls 138 and 139, which form a single sidewall such as a cylindrical sidewall in some embodiments. The base 106 engages, and may also be coupled to, the chamber 104 via an engagement 128. The engagement 128 is a fixed connection in some embodiments. In other embodiments the engagement 128 is a releasable engagement, and the base 106 can be considered a form of a cover that releasably engages the chamber 104 and seals a vaporization substance in the chamber 104. In such embodiments, the engagement 128 may include a threaded engagement or connection or an abutment between the chamber 104 and the base 106, for example. A gasket or other sealing member may be provided between the chamber 104 and the base 106 to seal the vaporization substance in the chamber. Such a releasable engagement enables removal or disengagement of the base 106 from the chamber 104 to permit access to the interior of the chamber, so that the chamber can be emptied, cleaned, and/or filled with a vaporization substance, for example. The base 106 is then re-engaged with the chamber 104 to seal the vaporization substance inside the chamber.

The base 106 generally includes circuitry to supply power to the atomizer 130. For example, the base 106 may include electrical contacts that connect to corresponding electrical contacts in the battery compartment 108. The base 106 may further include electrical contacts that connect to corresponding electrical contacts in the atomizer 130. The base 106 may reduce, regulate or otherwise control the power/voltage/current output from the battery compartment 108. However, this functionality may also or instead be provided by the battery compartment 108 itself. The base 106 may be made from one or more materials including metals, plastics, elastomers and ceramics, for example, to carry or otherwise support other base components such as contacts and/or circuitry. However, other materials may also or instead be used.

The combination of a cap 102, a chamber 104, a stem 110, an atomizer 130, and a base 106 is often referred to as a cartridge or “cart”.

The battery compartment 108 could also be referred to as a battery housing. The battery compartment 108 includes sidewalls 140 and 141, a bottom 142 and a button 144. The sidewalls 140 and 141, as noted above for other sidewalls, form a single wall such as a cylindrical sidewall in some embodiments. The battery compartment 108 engages, and may also couple to, the base 106 via an engagement 146. The engagement 146 is a releasable engagement in some embodiments, such as a threaded connection or a magnetic connection, to provide access to the inside of the battery compartment 108. The battery compartment 108 may include single-use batteries or rechargeable batteries such as lithium-ion batteries. A releasable engagement 146 enables replacement of single-use batteries and/or removal of rechargeable batteries for charging, for example. In some embodiments, rechargeable batteries are recharged by an internal battery charger in the battery compartment 108 without removing them from the vaporization device 100. A charging port (not shown) may be provided in the bottom 142 or a sidewall 140, 141, for example. The battery compartment 108 may be made from the same material(s) as the base 106 or from one or more different materials.

The button 144 is one example of a user input device, which may be implemented in any of various ways. Examples include a physical or mechanical button or switch such as a push button. A touch sensitive element such as a capacitive touch sensor may also or instead be used. A user input device need not necessarily require movement of a physical or mechanical element.

Although shown in FIG. 1 as a closed or flush engagement, the engagement 146 between the base 106 and the battery compartment 108 need not necessarily be entirely closed. A gap between outer walls of the base 106 and the battery compartment 108 at the engagement 146, for example, may provide an air intake path to one or more air holes or apertures in the base that are in fluid communication with the interior of the stem 110. An air intake path may also or instead be provided in other ways, such as through one or more apertures in a sidewall 138, 139, elsewhere in the base 106, and/or in the battery compartment 108. When a user draws on a mouthpiece, air is pulled into the air intake path and through a channel. In FIG. 1, the channel runs through the atomizer 130, where air mixes with vapor formed by the atomizer, and the stem 110. The channel also runs through the cap 102 in some embodiments.

The battery compartment 108 powers the vaporization device 100 and allows powered components of the vaporization device, including at least the atomizer 130, to operate. Other powered components could include, for example, one or more light-emitting diodes (LEDs), speakers or other elements to provide indicators of, for example, device power status (on/off), device usage status (on when a user is drawing vapor), etc. In some embodiments, speakers and/or other elements generate audible indicators such as long, short or intermittent “beep” sounds as a form of indicator of different conditions. Haptic feedback could also or instead be used to provide status or condition indicators. Varying vibrations and/or pulses, for example, may indicate different statuses or actions in a vaporization device, such as on/off, currently vaporizing, power source connected, etc. Haptic feedback may be provided using small electric motors as in devices such as mobile phones, other electrical and/or mechanical means, or even magnetic means such as one or more controlled electronic magnets.

As noted above, in some embodiments, the cap 102, the chamber 104, the stem 110, the atomizer 130, the base 106 and/or the battery compartment 108 are cylindrical in shape or otherwise shaped in a way such that sidewalls that are separately labeled in FIG. 1 are formed by a single sidewall. In these embodiments, the sidewalls 114 and 115 represent sides of the same sidewall. Similar comments apply to outer walls 118 and 120, sidewalls 131 and 133, outer walls 122 and 124, sidewalls 138 and 139, sidewalls 140 and 141, and other walls that are shown in other drawings and/or described herein. However, in general, caps, chambers, stems, atomizers, bases and/or battery compartments that are not cylindrical in shape are also contemplated. For example, these components may be rectangular, triangular, or otherwise shaped.

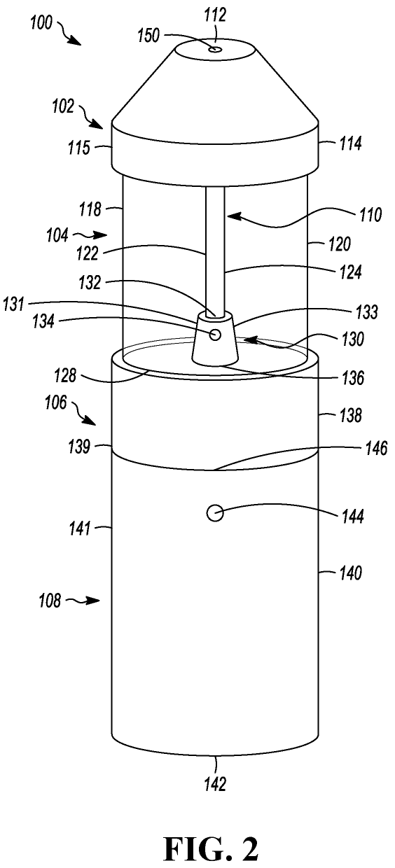

FIG. 2 is an isometric view of the vaporization device 100. In FIG. 2, the cap 102, the chamber 104, the stem 110, the atomizer 130, the base 106 and the battery compartment 108 are illustrated as being cylindrical in shape. As noted above, this is not necessarily the case in other vaporization devices. FIG. 2 also illustrates a hole 150 through the tip 112 in the cap 102. The hole 150 is coupled to the stem 110 through a channel in the cap 102. The hole 150 allows a user to draw vapor through the cap 102. In some embodiments, a user operates the button 144 to vaporize a vaporization substance for inhalation through the cap 102. Other vaporization devices are automatically activated, to supply power to powered components of the vaporization device when a user inhales through the hole 150. In such embodiments, a button 144 need not be operated to use a vaporization device, and need not necessarily even be provided at all.

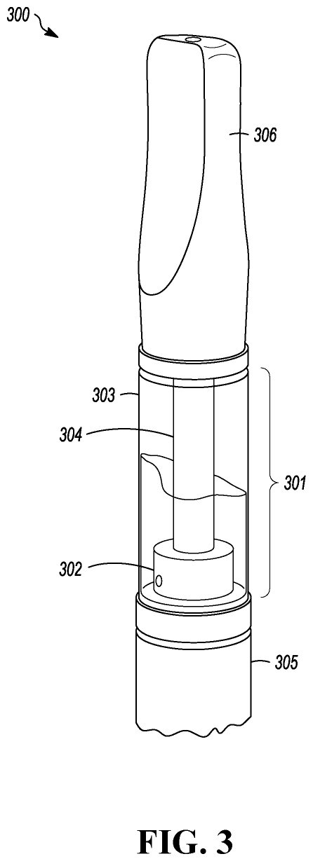

FIG. 3 is an isometric view of another example vaporization device 300. Reference number 301 in FIG. 3 generally designates a vape tank, with a ceramic core 302 coupled to a chamber 303 that stores a vaporization substance. The vape tank 301 is powered by a power source (e.g. battery) inside a compartment 305 that physically and electrically connects to the vape tank. In some implementations, the vaporization device 300 has a control system (not shown) for controlling how the power source provides power to the vape tank 301.

During use, the vaporization substance from the chamber 303 seeps into the ceramic core 302, which heats the vaporization substance using a heating element (not shown) enough to atomize the vaporization substance, thereby producing vapor. The vapor can be drawn out of the ceramic core 302 through a stem 304 and out of the vaporization device 300 through a mouthpiece 306. The structure and operation of the vaporization device 300 are consistent with those of the example vaporization device 100 in FIGS. 1-2, and is presented as a further example to illustrate another shape and form factor of a vaporization device. Embodiments of the present disclosure may be implemented in conjunction with these and/or other types of vaporization devices.

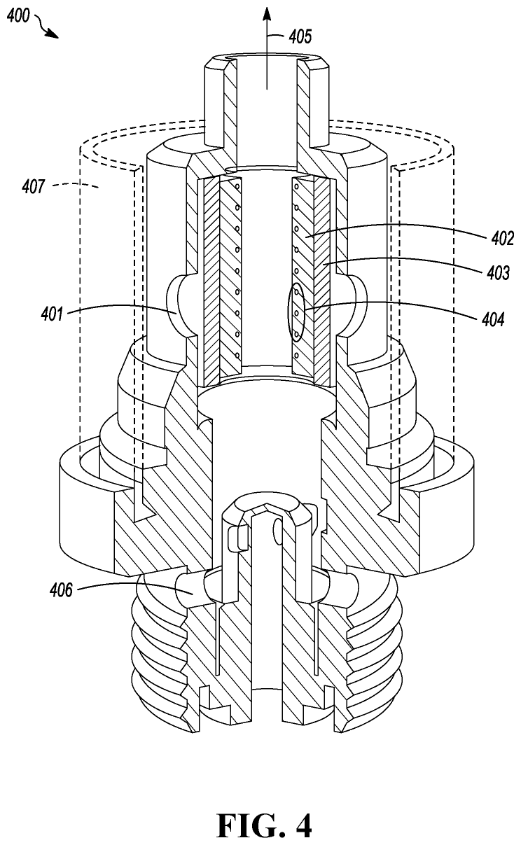

FIG. 4 is a diagram illustrating internal structure of an example vaporization device tank 400 with a ceramic core 402. The example vape tank 400 is shown with a section removed so that internals of the vape tank can be seen. The vape tank 400 can be implemented in a vaporization device, non-limiting examples of which are shown in FIGS. 1 to 3. It is to be understood that the vape tank 400 is a very specific example and is provided for illustrative purposes only.

In some implementations, as shown in the illustrated example, the vape tank 400 has an inlet 401 for receiving a vaporization substance from a chamber 407. In other implementations, there is no such inlet 401 or chamber 407, and the vaporization substance is supplied to the ceramic core 402 by other means such as manual application by a user for example. The ceramic core 402 has a heating element 404 embedded therein. A physical characteristic of the ceramic core 402, such as density or porosity, enables the vaporization substance to seep through the ceramic core, particularly when the vaporization substance has been heated by the heating element 404 to reduce its viscosity.

In some implementations, the vape tank 400 has an element or component to feed the vaporization substance to the ceramic core 402. An example of such an element or component is a wick as shown at 403, disposed between the chamber 407 and the ceramic core 402. In some implementations, the wick 403 is made from cotton or any other suitable material that has a lower porosity than the ceramic core 402. In some implementations, the porosity of the wick 403 is high enough that the vaporization substance can easily seep through and make contact with the ceramic core 402 even without any heating from the heating element 404 embedded in the ceramic core. The wick 403 may help provide more even contact between the vaporization substance and the ceramic core 402. In other implementations, a vape tank has no such wick 403.

In some implementations, the heating element 404 is a coil heater with a number of coil turns or loops embedded in the ceramic core 402. Three of these coil turns or loops are identified by an oval in the illustrated example, but more coil turns or loops are visible in FIG. 4. The number of coil turns or loops is implementation-specific. Other examples of heaters or heating elements are also provided herein.

The coil heater 404 is embedded into the ceramic core 402 during manufacture of the ceramic core in some embodiments. The ceramic core 402 has a heat capacity, and thus embedding the coil turns or loops in the ceramic core can help to avoid a situation in which the coil turns or loops directly contact the vaporization substance and become too hot, burning rather than vaporizing the vaporization substance or at least certain components of the vaporization substance.

In some implementations, the heating element 404 is positioned closer to an inside or interior portion of the ceramic core 402 and closer to the channel 405 as shown, such that the vaporization substance may reach progressively higher temperatures as it seeps through the ceramic core towards the channel. When the vaporization substance seeping through the ceramic core 402 is sufficiently heated, it is atomized to produce a vapor, which can be drawn out through the channel 405. In other implementations, the heating element 404 is positioned in a middle portion of the ceramic core 402. In other implementations, the heating element 404 is positioned outside of the ceramic core 402 and around or in the channel 405.

The temperature at which the vaporization substance is atomized to produce the vapor may depend on any one or more of a number of factors such as the vaporization substance being used, thermal conductivity of the ceramic core 402, and/or thermal conductivity of the vaporization substance itself. As a specific example, the temperature at which the vaporization substance is atomized may be around 300° F. or higher. In a specific example, the temperature of the vaporization substance should not exceed 600° F. or else it may burn.

During use, the heating element 404 heats up the ceramic core 402 and generates vapor by atomizing the vaporization substance seeping through the ceramic core. The vapor can be drawn out through a channel 405, and an air inlet 406 is disposed beneath the ceramic core 402 to facilitate airflow for the channel 405. In some implementations, the heating element 404 is powered by a power source (not shown) and controlled by a control system (not shown). In some implementations, the power source and the control system are disposed in a compartment that physically and electrically connects to the vape tank 400. Such connections include electrical connections (not shown) between the heating element 404 and the power source and/or the control system.

Although the channel 405 is labelled at the top of the view shown in FIG. 4, it should be appreciated that embodiments disclosed herein may be implemented in any of various sections or parts of a channel 405, including any one or more of: downstream from the ceramic core 402 in a direction of air flow during use of a vaporization device, which is above the ceramic core 402 in the view shown in FIG. 4, such as in the stem or chimney of a vaporization device; within a section or part of the channel that passes through or along the ceramic core 402; and upstream from the ceramic core 402 in a direction of air flow during use of a vaporization device, which is below the ceramic core 402 in the view shown in FIG. 4, such as in an intake section toward the air inlet 406.

Some aspects of the present disclosure relate to vaporization devices that include a cooler to reduce the temperature of vapor prior to inhalation by a user. As noted above, high temperature vapor may cause discomfort and irritation for the user. Thus, cooling the vapor prior to inhalation could provide a more pleasurable and safe experience for the user. A cooler could allow for an atomizer to operate at relatively high temperatures to produce a high quantity and/or quality of vapor, while still providing a user with vapor that is at a comfortable temperature.

Adding a cooler to a vaporization device could be considered counter-intuitive to conventional wisdom. Vaporization devices typically involve heating vaporization substances to achieve vaporization, and implementing coolers would appear to counteract this principle. However, as noted above, vapor cooling can provide potential advantages in terms of user comfort, for example.

Vapor cooling in a vaporization apparatus could be achieved in any of variety of different ways. In some embodiments, vapor cooling includes the process of reducing the temperature of the vapor by transferring heat away from the vapor. Vapor cooling could also or instead include the process of mixing a vapor with another lower-temperature fluid, such as air for example, which produces a mixture with a lower temperature.

It should be noted that although at least some loss of heat from vapor is expected in a vaporization device, as vapor flows through a channel and heat is transferred to the walls of the channel for example, this transfer of heat typically does not lead to a substantial decrease in vapor temperature. A channel in a conventional vaporization device might not sufficiently reduce the temperature of a hot vapor to provide a vapor that is pleasant for a user to inhale. Therefore, at least in this sense, a channel in a conventional vaporization device is not considered to be a cooler in the context of the present disclosure.

Vapor cooling can be defined using any of a variety of metrics. For example, vapor cooling can be defined in terms of achieving a target vapor temperature for inhalation. This temperature can be user-defined, can be based on properties of a vaporization substance, and/or can be predetermined based on a vapor temperature that is expected to be pleasant for a user. Vapor cooling can also or instead be defined in terms of a finite or relative temperature drop for the vapor in a cooler. An example of a cooler with a finite temperature drop is a device that provides a temperature decrease of approximately 50° C. An example of a cooler with a relative temperature drop is a device that provides an approximately 25% reduction in vapor temperature. Vapor cooling may also or instead be defined or quantized in terms of a range of temperature change, such as at least a particular finite temperature change, within a range of finite temperature changes, at least a particular relative temperature change, and/or within a range of relative temperature changes. For example, vapor cooling could be defined or quantized as lowering temperature of vapor by 5-15% or 1-15%. Other ranges, definitions, or quantizations are possible.

Cooling a vapor can potentially result in condensation of the vapor. Condensation in a channel, mouthpiece or any other component of the vaporization device could reduce the quantity and/or quality of vapor that is available for a user to inhale. Condensation of vapor in a vaporization device may also or instead lead to leakage that is messy and annoying for a user. However, as discussed elsewhere herein, vapor cooling can be achieved without causing substantial levels of condensation in some embodiments.

Temperatures at which a vapor may condense or deposit inside a vaporization device could be different from vaporization temperatures. Therefore, target temperatures for cooling could be determined based on condensation and/or deposition temperature(s). In some embodiments, a cooler has a form of temperature control to maintain vapor temperature above the condensation temperature of the vapor. Different vaporization substances or ingredients therein could have different condensation temperatures, and target temperatures for cooling could be determined based on the condensation temperature(s) of the particular vaporization substance(s) or ingredient(s) that are in a vapor.

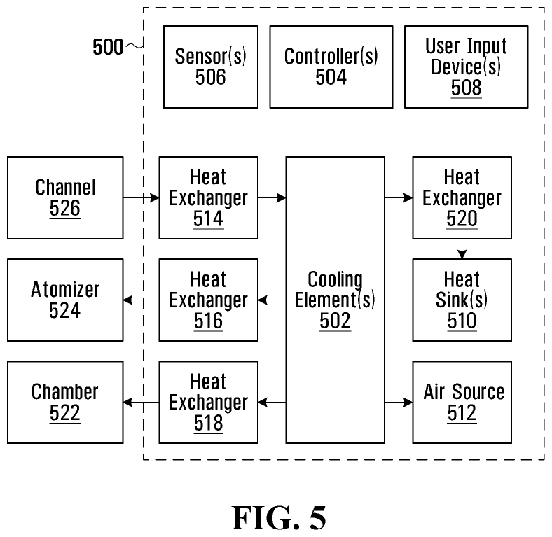

Reference will now be made to FIGS. 5 to 20, which provide various examples of coolers to cool vapor in a vaporization apparatus such as a cap, mouthpiece, cartridge or vaporization device. These examples are merely meant to be illustrative, and should not be considered limiting in any way.

FIG. 5 is a block diagram illustrating a cooler 500 according to an embodiment. The cooler 500 includes one or more cooling element(s) 502, one or more controller(s) 504, one or more sensor(s) 506, one or more user input devices(s) 508, one or more heat sink(s) 510, a source of air 512, and multiple heat exchangers 514, 516, 518, 520. The arrows in FIG. 5 represent heat transfer and not necessarily physical connections or couplings between components.

FIG. 5 also illustrates a chamber 522 to store a vaporization substance, an atomizer 524 to generate vapor from the vaporization substance by heating the vaporization substance, and a channel 526 to carry the vapor away from the atomizer. The atomizer 524 is in fluid communication with the chamber 522, and the channel 526 is in fluid communication with the atomizer 524. The chamber 522, atomizer 524 and/or channel 526 could be similar to any chamber, atomizer or channel described above with reference to FIGS. 1 to 4.

In some implementations, the cooler 500, the chamber 522, the atomizer 524 and the channel 526 are components of a vaporization device. The vaporization device could also include a mouthpiece (not shown) to enable inhalation of the vapor by a user. The mouthpiece could at least partially include the channel 526 and/or the cooler 500.

A box with a dashed line is drawn around the various elements of the cooler 500 in FIG. 5. This box is merely provided for illustrative purposes, and is not intended to limit how the cooler 500 could be implemented. It should be noted that the cooler 500 could be implemented as a single component in a vaporization device, or multiple components distributed at different locations in a vaporization device. Electrical and/or fluidic connections could be provided between any or all of the multiple components. Although the cooler 500 is illustrated at being separate from the chamber 522, atomizer 524 and channel 526, the cooler or any component thereof could instead be integrated or coupled with the chamber, atomizer and/or channel.

The arrows shown in FIG. 5 illustrate possible transfers of heat to, within and away from the cooler 500. For example, the arrow between the cooling element(s) 502 and the source of air 512 indicates that heat could be transferred from one or more cooling elements to the air. These arrows are merely provided by way of example, and are not intended to be limiting. Others coolers could transfer heat in different ways.

The cooling element(s) 502 are provided to cool the vapor through conduction, convection, radiation, and/or some other effect. Heat conduction could also be referred to as heat diffusion. In some implementations, one or more of the cooling element(s) 502 are in fluid communication with, and optionally located at least partially inside of, the channel 526 to directly cool the vapor. Vapor cooling could also or instead be indirect. For example, one or more of the cooling element(s) 502 could cool a component through which the vapor flows, such as a stem, cap or mouthpiece that defines the channel 526.

The cooling element(s) 502 could be active and/or passive. A passive cooling element as referenced herein is a cooling element that includes no powered or controlled components, whereas an active cooling element includes one or more powered and/or controlled components.

An example of a passive cooling element is a thermal conductor to transfer heat away from the vapor. For example, the thermal conductor could be implemented inside of the channel 526, and/or be implemented in contact with a structure defining the channel, to conduct heat away from the vapor. The thermal conductor could include any material that allows energy in the form of heat to be transferred. Examples of these materials include metals with relatively high thermal conductivities such as copper, graphene, graphite, silver, gold and aluminium. A passive cooling element could also include a fluid, stored in a chamber, which can transfer heat via convection in the chamber, for example.

Although shown separately in FIG. 5, a heat sink is another example of a passive cooling element.

In some embodiments, a passive cooling element includes a thermal conductor having a thermal conductivity that increases with temperature. This could also be referred to as a temperature-dependent thermal conductivity. When a high temperature vapor comes into contact with this thermal conductor, or otherwise transfers heat to the thermal conductor, the temperature and thermal conductivity of the thermal conductor both increase. After a period of time, the temperature and thermal conductivity of the thermal conductor could be relatively high. This could result in heat being conducted away from the vapor relatively rapidly, and therefore the temperature of the vapor could decrease relatively rapidly as a result. As the temperature of the vapor decreases, the temperature and thermal conductivity of the thermal conductor might also decrease. This then reduces the rate at which heat is conducted away from the vapor, and also reduces the rate at which the temperature of the vapor decreases. A potential benefit of such temperature-dependent thermal conductivity is to help prevent over-cooling a vapor. If a vapor is cooled to temperatures that are below a certain threshold, a high rate of condensation could occur. This condensation could degrade the quantity and/or quality of the vapor, and potentially result in build-up of the vaporization substance in a channel. Accordingly, thermal conductors having a thermal conductivity that increases with temperature could be considered to provide a form of passive temperature control or regulation.

Examples of an active cooling element include a thermoelectric cooler or cooling element, fluid regulators, fans and/or coolant pumps, for example. A thermoelectric cooling element is an electrically powered cooling element that typically includes a “cold side” and a “hot side”. The cold side could be used to cool vapor in a vaporization device. The hot side could also be used in a vaporization device to help heat and/or vaporize a vaporization substance, for example. As such, a thermoelectric cooling element could be implemented as a dual-purpose heating and cooling element in a vaporization device, examples of which are provided elsewhere herein.

In some embodiments, active cooling elements are powered by a battery and/or another power source. This power source could also power other components of a vaporization device such as an atomizer, for example. To enable control of an active cooling element in the cooler 500, the active cooling element could be coupled to, or otherwise configured to interface with, the controller(s) 504, the sensor(s) 506 and/or the user input device(s) 508.

The sensor(s) 506 could include temperature sensors that are provided to measure the temperature of vapor at any location in a vaporization device. Non-limiting examples of temperature sensors include thermocouples and thermistors. In some implementations, at least one of the sensor(s) 506 is provided to measure vapor temperature in the channel 526. The sensor could be located inside of the channel 526 to directly measure the temperature of vapor inside of the channel, or the sensor could be coupled to an outer wall of the channel to indirectly measure the temperature of the vapor, for example. One or more temperature sensors could also or instead be provided to measure temperatures in the chamber 522, the atomizer 524 and/or any other component of a vaporization device. In addition to or instead of temperature sensors, the sensor(s) 506 could include other types of sensors such as pressure sensors, for example.

Although illustrated as components of the cooler 500, any or all of the sensor(s) 506 could instead be separate from the cooler.

The user input device(s) 508 could include buttons, switches, sliders, dials, and/or other types of input device that enable a user to control any of various aspects or parameters of the cooler 500. For example, if a user wishes to reduce the temperature of the vapor, the user could manipulate one or more of the user input device(s) 508 to initiate or increase cooling by the cooler 500. The user could also manipulate one or more of the user input device(s) 508 to stop or decrease cooling by the cooler 500.

In some implementations, the user input device(s) 508 enable user control of the cooler 500 as well as other components of a vaporization device. For example, the user input device(s) 508 could enable user control over the operation of the atomizer 524. Accordingly, any or all the user input device(s) 508 might not be specific or dedicated only to the cooler 500, and could also provide user inputs to one or more other components of a vaporization device.

The controller(s) 504 could be implemented, for example, using hardware, firmware, one or more components that execute software stored in one or more non-transitory memory devices (not shown), such as a solid-state memory device or a memory device that uses movable and/or even removable storage media. Microprocessors, Application Specific Integrated Circuits (ASICs), Field Programmable Gate Arrays (FPGAs), and Programmable Logic Devices (PLDs) are examples of processing devices that could be used to execute software. In some implementations, the controller(s) 504 control the cooler 500 as well as other components of a vaporization device. For example, the controller(s) 504 could control the operation of the atomizer 524. Accordingly, any or all the controller(s) 504 may not be specific or dedicated only to the cooler 500, and could also provide or enable control over one or more other components of a vaporization device.

In some implementations, the controller(s) 504 are in communication with any or all of the cooling element(s) 502, the sensor(s) 506 and the user input device(s) 508. For example, the controller(s) 504 could control one or more of the cooling element(s) 502 responsive to a measurement of the temperature of the vapor received from the sensor(s) 506, and/or responsive to input received from a user by the user input device(s) 508. To control an active cooling element, for example, the controller(s) 504 could adjust the power delivered to the active cooling element from a power source. In some implementations, the user input device(s) 508 allow a user to input a desired vapor temperature, and the sensor(s) 506 provide feedback to the controller(s) 504 to help achieve that vapor temperature. In some implementations, the controller could determine the condensation temperature of the vapor, and the sensor(s) 506 provide feedback to the controller(s) 504 to help maintain vapor temperature above the condensation temperature. Examples of how a condensation temperature of a vaporization substance can be determined are discussed elsewhere herein. In some implementations, one or more sensor(s) 506 detect the condensation of vapor in the channel 526, and the controller(s) 504 decrease cooling in response to detection of condensation.

The heat sink(s) 510 are provided to absorb heat from the vapor. In some implementations, one or more of the heat sink(s) 510 is/are initially at or below ambient temperature before receiving heat from the vapor. A heat sink that is below ambient temperature could be provided by refrigerating the heat sink before use, for example. The heat sink(s) 510 could have a much larger thermal mass than the vapor, and therefore the temperature of the heat sink(s) could increase at a relatively slow rate as heat is received from the vapor. In some implementations, a heat sink is made from one or more materials that have a high heat capacity compared to the vapor. This may help provide a consistent rate of cooling by the cooler 500.

A heat sink 510 may include any of various materials. Any of solids, liquids and gases could be implemented in a heat sink. For example, a heat sink could be a hollow component with a gas such as air or a liquid such as water inside. In some implementations, one or more of the heat sink(s) 510 include a material that is continuously or repeatedly replaced or cycled. For example, a heat sink may be coupled to the source of air 512, or any other source of fluid, which replaces a fluid in the heat sink.

In some implementations, a heat sink is made from or includes a phase change material. For example, a heat sink may include a phase change material with a melting point that is below the temperature of vapor exiting an atomizer, above typical ambient temperatures, and corresponding to a desired vapor temperature for inhalation. The phase change material could also or instead have a high heat of fusion, and therefore a relatively large amount of heat may be required to melt the phase change material. As the phase change material absorbs heat, it may remain near this melting point temperature, and thus enable a heat sink to absorb heat at a steady rate even after repeated and/or prolonged use of the heat sink to cool vapor. Non-limiting examples of phase change materials include paraffin waxes and hydrated salts.

The source of air 512 may be used to mix air and vapor and thereby reduce the temperature of the vapor in the resulting mixture. In some implementations, air is drawn from the ambient atmosphere. For example, the source of air 512 could include an air inlet and/or an air intake channel that is/are in fluid communication with the channel 526. The air intake channel may, at least in part, transfer air from the ambient atmosphere to the channel 526 to mix the vapor with air and cool the vapor. In such an implementation, the air intake channel could be considered a passive cooling element. Active cooling elements, such as fans and regulators for example, may be implemented at or in an air intake channel to control the flow of air. Moreover, thermoelectric coolers may be implemented at or in in an air intake channel to reduce the temperature of the air and further cool the vapor.

In some implementations, the source of air 512 is a compressed air reservoir such as a cylinder. A compressed air cylinder may be compact so as to not substantially increase the size and weight of a vaporization device, and may be controllable to release air and mix the air with vapor to cool the vapor. Compressed air expands and cools as it leaves a cylinder, thereby providing a supply of cool air that may be below ambient temperatures. This could reduce the amount of air that is needed to cool the vapor, at least when compared to air at ambient temperatures. The resulting mixture of vapor and air could be less diluted as a result.

In should be noted that although air is one example of a gas that can be used to mix with and/or cool a vapor, other gases are also contemplated. For example, compressed nitrogen could be implemented in a cooler.

A heat exchanger is a device to transfer heat from one medium to another. In the cooler 500, the heat exchangers 514, 516, 518, 520 are provided to transfer heat to, from and/or within the cooler. In some implementations, any or all of the heat exchangers 514, 516, 518, 520 use a thermally conductive material to transfer heat. In some cases, this thermally conductive material is also be considered a passive cooling element.

In some implementations, any or all of the heat exchangers 514, 516, 518, 520 use one or more fluids, such as air or a liquid for example, to transfer heat. Non-limiting examples of such heat exchangers include shell and tube heat exchangers, and plate heat exchangers. A fluid in a heat exchanger may be referred to as a coolant or refrigerant, for example. One or more of the heat exchangers 514, 516, 518, 520 may include a closed system with a coolant being pumped or circulated between various components of the cooler 500 and/or a vaporization device. A heat exchanger may instead be an open system that receives a repeated or continuous supply of coolant. For example, a heat exchanger may circulate air from the source of air 512 to the channel 526 to receive heat from the vapor. The air may then be circulated to another component of a vaporization apparatus, or be expelled into the atmosphere.

Although illustrated as separate elements, any two or more of the heat exchangers 514, 516, 518, 520 may instead be implemented as a single element.

The heat exchanger 514 is provided to transfer heat away from the channel 526, which may include transferring heat away from the vapor in the channel. In some implementations, the heat exchanger 514 carries a coolant inside of, and/or adjacent to, the channel 526 to collect heat from the vapor and/or the walls of the channel 526. Optionally, the heat exchanger 514 may include a tube carrying coolant inside the channel 526. Although the heat exchanger 514 is illustrated as transferring heat to the cooling element(s) 502, the heat exchanger 514 may also or instead transfer heat to other components of a cooler and/or vaporization device, such as a heat sink for example.

The heat exchanger 516 is provided to transfer heat to the atomizer 524, and the heat exchanger 518 is provided to transfer heat to the chamber 522. In some implementations, the cooling element(s) 502 provide heat that is transferred by the heat exchangers 516, 518. For example, heat from the hot side of a thermoelectric cooler may be transferred to the atomizer 524 and/or to the chamber 522 by the heat exchangers 516, 518. Although not shown in FIG. 5, heat that is transferred by the heat exchangers 516, 518 could also or instead come directly from the vapor in the channel 526.

The heat exchangers 516, 518 provide possible mechanisms for re-use of heat from the vapor. Heat that is transferred by the heat exchanger 516 could be used by the atomizer 524 to help vaporize the vaporization substance. As a result, the atomizer 524 may draw less power from a power source to achieve vaporization. Heat that is transferred by the heat exchanger 518 could be used to heat the vaporization substance in the chamber 522. As a result, the viscosity of the vaporization substance could be decreased, and the vaporization substance may flow into the atomizer 524 more rapidly. For example, the reduced viscosity of the vaporization substance could enable the vaporization substance to flow through a ceramic core more rapidly. This may be considered a form of pre-treatment or priming of the vaporization substance. In addition, the heated vaporization substance may be less prone to clinging or adhering to the walls of the chamber 522 and being wasted.

The heat exchanger 520 is provided to transfer heat to the heat sink 510. In some implementations, the heat sink comprises a material in the heat exchanger. For example, the heat exchanger 520 may circulate a fluid to carry heat away from one or more of the cooling element(s) 502, where the fluid also functions as a heat sink.

Although the heat exchangers 514, 516, 518, 520, heat sink(s) 510 and the source of air 512 may be considered forms of active and/or passive cooling elements, they are shown separately from the cooling element(s) 502 to better illustrate how multiple components can be integrated in a cooler.

One or more components of the cooler 500, and even the cooler itself, could be removable or replaceable in a vaporization device. For example, one or more of the cooling element(s) 502 or heat sink(s) 510 could be a removable cooling element that is releasably coupled to a vaporization device. Such a removable cooling element could allow for a certain degree of control over cooling, as a user could add or remove the cooling element to achieve a desired vapor temperature. In some implementations, the cooler 500 or any removable cooling element is coupled to the vaporization apparatus by a releasable coupling. For example, a cooler or removable cooling element could be coupled to a vaporization apparatus magnetically.

The cooler 500 is illustrative of one possible implementation of sensor(s), controller(s), user input device(s), heat exchangers, cooling element(s), heat sink(s) and a source of air. Other embodiments with more or fewer elements and/or different arrangements of elements are also contemplated.

One or more of the heat exchangers 514, 516, 518, 520 could be excluded in other coolers. For example, a cooling element could be placed inside of, and/or in direct contact with, a channel to receive heat from the channel and/or the vapor in the channel. For example, air from the source 512 is mixed directly with vapor in some embodiments, and accordingly no heat exchanger is shown for the air source.

The cooling element(s) 502 could be excluded in other coolers. Heat from a vapor could instead be transferred directly to a heat sink, for example. Optionally, a heat exchanger could be used to help facilitate the transfer of heat from the vapor. The heat sink(s) 510 and/or the source of air 512 could also or instead be excluded in other coolers.

Condensation may be stimulated when a vapor is cooled below its condensation temperature by a cooler. Accordingly, some embodiments provide features to prevent, inhibit or limit condensation in a vaporization device. Condensation generally occurs through heterogeneous nucleation of vapor on the surface of a different substance, such as the wall of a channel or a particle of an impurity. Thus, vapor condensation can be kinetically inhibited by minimizing the amount of solid particles in the vapor, and the surface area with which the vapor comes into contact.

In some embodiments, condensation is reduced or managed by minimizing the length of a channel through which the vapor travels and/or by smoothing the inner surfaces the channel. For example, a cooler such as the cooler 500 could be implemented inside or proximate to a mouthpiece to limit the distance a cooled vapor travels before inhalation by a user. As such, a relatively high temperature vapor can be cooled prior to inhalation with little to no condensation in the vaporization device.

Moreover, to extent that the coolers and/or other features disclosed herein might lead to condensation of vapor and the formation of liquid in a vaporization device, features such as those disclosed in U.S. Provisional Application No. 62/896,225, filed on Sep. 5, 2019, incorporated in its entirety herein by reference, may be implemented to manage liquid in the vaporization device.

FIG. 5 is a general block diagram representation of an example cooler. Other examples are provided at least below.

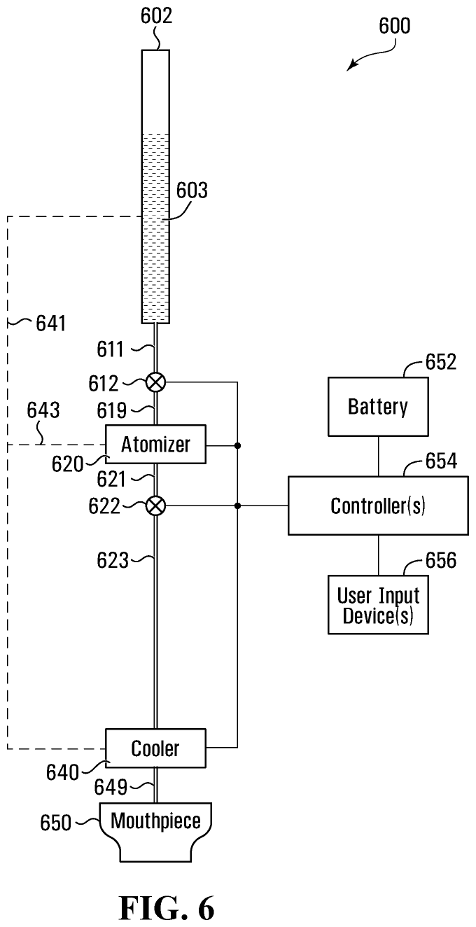

FIG. 6 is a block diagram illustrating an example vaporization device 600. The vaporization device 600 includes a chamber 602 to store a vaporization substance 603. The chamber 602 could be similar to the chamber 104 described above with reference to FIGS. 1 and 2, for example. The chamber 602 could include an engagement structure to engage with a complementary engagement structure of the example device 600. These engagement structures could limit the vaporization device 600 to certain types of chambers.

The chamber 602 could be recloseable or non-recloseable. Examples of releasable engagements for recloseable chambers and non-releasable engagements for non-recloseable chambers are provided elsewhere herein.

An atomizer 620 is in fluid communication with the chamber 602 through channels 611, 619 and a valve 612 in the example shown. The valve 612 is an example of a regulator to control movement of the vaporization substance 603 from the chamber 602. Other forms of regulators include, for example, wicks, pumps, and mechanical feed structures such as screw conveyors and spray nozzles to spray vaporization substances into the atomizer 620.

Regardless of the type of regulator, a regulator may be useful in providing a measure of dosage control. Dosage of an active ingredient in the vaporization substance 603, for example, could be controlled by controlling the valve 612.

The valve 612 is in fluid communication with the atomizer 620 through the channel 619. In some embodiments, the valve 612 could be integrated with the atomizer 620 in a single component. The valve 612 controls the movement of the vaporization substance 603 to the atomizer 620, which generates a vapor by heating the vaporization substance. The atomizer 620 includes a heater to heat the vaporization substance. The heater could include, for example, a coil heater, a fan heater, a ceramic core heater, and/or a quartz heater. The atomizer 620 could be implemented as described above with reference to FIGS. 1 to 4.

The vapor produced by the atomizer 620 is fed into a channel 621. The channel 621 is in fluid communication with the atomizer 620, to carry the vapor away from the atomizer. The vapor valve 622 is an example of a vapor regulator, which is provided to control a flow of the vapor from the atomizer 620.

The vaporization device 600 also includes a cooler 640, in fluid communication with the atomizer 620 through channels 621, 623 and the vapor valve 622 in the embodiment shown, to cool the vapor before inhalation by a user. In some implementations, the cooler 640 is similar to the cooler 500 of FIG. 5. The cooler 640 could include any number and arrangement of sensors, controllers, user input devices, heat exchangers, cooling elements, heat sinks and sources of air.

In some implementations, at least a portion of the cooler 640 is located at a position inside a channel, such as the channel 623, to cool vapor directly. The cooler 640 could also or instead be implemented outside a channel or air flow path, by being coupled to or in contact with an outside wall of the channel 623 for example, to indirectly cool the vapor by cooling one or more components through which the vapor flows. The cooler 640 could be coupled to a channel by adhesives or fasteners, for example. More generally, one or more coolers could be implemented to cool the vapor that is generated by the atomizer 620, and/or to cool one or more device components.

The cooled vapor is carried to a mouthpiece 650 by a channel 649. The mouthpiece 650 is in fluid communication with the atomizer 620, with the cooler 640, and with the channels 621, 623, 649 therebetween. The mouthpiece 650 enables inhalation of the vapor by the user. In general, the mouthpiece 650 could be directly or indirectly in fluid communication with other components. The channel 649, for example, could be a hose or other channel through which the mouthpiece 650 is indirectly in fluid communication with other components of the vaporization device 600. Like other channels in FIG. 6, the channel 649 could include one or more vapor regulators.

The position of the cooler 640 relative to the mouthpiece 650 and/or the atomizer 620 is implementation specific. In some cases, the position of the cooler 640 is based on an expected temperature of the vapor in either or both of the channels 623, 649. For example, if the vapor is cooled by the cooler 640 to a temperature that is below a condensation temperature of the vapor, then the cooler may be positioned proximate to, or even in contact with, the mouthpiece 650 to limit the length of the channel through which the cooled vapor will travel after cooling. This could limit condensation of vapor in the channel 649 and/or in the mouthpiece 650.

In some implementations, the mouthpiece 650 at least partially includes the cooler 640. For example, at least a portion of the cooler 640, and/or another cooler, is located at a position inside the mouthpiece 650 to provide vapor cooling. The cooler 640, and/or another cooler, could also or instead be coupled to the mouthpiece. In general, the cooler 640 may be provided in fluid communication with the channel 649 upstream of the mouthpiece 650, and/or within the mouthpiece.

In some embodiments, the channel 649 and/or the mouthpiece 650 also or instead provide vapor cooling. Characteristics such as length and/or material composition of the channel 649, which could be or include a mouthpiece hose for example, could be selected to provide for cooling of vapor. A longer channel 649 provides more time for vapor to cool before reaching the mouthpiece 650 and being inhaled by a user. A channel and/or mouthpiece that is made from or at least includes one or more thermally conductive material(s) could provide or improve vapor cooling prior to inhalation.

Cooling could also or instead be provided by one or more additional air intakes, in the mouthpiece 650 and/or elsewhere in a vaporization device, to admit air into a vapor flow to cool the vapor. For example, in some implementations the cooler 640 provides one or more air intake channels in the mouthpiece 650 and/or elsewhere. Air is admitted into a vapor flow to cool the vapor, by the air intake channels in such implementations.

The dashed lines at 641, 643 illustrate optional heat transfers from the cooler 640 to the atomizer 620 and/or to the chamber 602. For example, the cooler 640 may include one or more heat exchangers that transfer heat from vapor to the atomizer 620 and/or to the chamber 602. Further examples of transferring heat from vapor to an atomizer and/or a chamber are provided elsewhere herein.

The valve 612, the atomizer 620, the vapor valve 622, and the cooler 640 are controlled by one or more controllers 654. A controller at 654 could be implemented, for example, using hardware, firmware, one or more components that execute software stored in one or more non-transitory memory devices (not shown), such as a solid-state memory device or a memory device that uses movable and/or even removable storage media. Examples of processing devices that could be used to execute software are provided at least above.

A power source such as a battery 652 and one or more user input devices 656 are coupled to the controller(s) 654. The user input device(s) 656 could include switches, sliders, dials, and/or other types of input device that enable a user to control any of various aspects or parameters of the valve 612, the atomizer 620, the vapor valve 622, and/or the cooler 640. Other input device examples are disclosed elsewhere herein, with reference to the button 144 in FIGS. 1 and 2 and the user input device(s) 508 in FIG. 5, for instance.

The battery 652 provides power to the controller(s) 654, which could then provide power to other components of the example device 600. The valve 612 and/or the vapor valve 622 could be controlled in this type of implementation by controlling power to the valve. For example, the valve 612 and/or the vapor valve 622 could be normally closed when not supplied with power, and opened when powered. In other embodiments, power and control are implemented separately. Other control mechanisms are also possible. However, not all types of regulators are necessarily controlled. A wick, for example, draws a vaporization substance from a chamber to an atomizer for vaporization, but the wick itself is not controlled.

A controller at 654 also controls and supplies power to the atomizer 620, and could provide on-off power control based on operation of a power button or switch at 656 or a user inhaling on the device 600, for example. In some embodiments, different voltages and/or currents could be supplied to the atomizer 620 to enable the atomizer to provide different temperatures for vaporization. This type of power control, which could be considered a form of temperature control, could be provided through a user input device 656, and/or based on sensing the type of chamber 602 currently installed in the device 600. For example, the chamber 602 could include an indicator of its vaporization substance 603. Using this indicator, a controller 654 could determine a vaporization temperature that is appropriate for the vaporization substance 603, and control the power delivered to the atomizer 620 accordingly. The voltage, current, and/or power supplied to the atomizer 620 could also or instead be controlled based on a desired flow or quantity of vapor produced by the atomizer, which could be selected or otherwise controlled using a user input device 656, for example.

A controller at 654, which could be the same controller that controls other components or a different controller, could control and power the cooler 640, including any or all active cooling elements in the cooler for example, to reduce vapor temperature. This control could be similar to the control of the atomizer 620 discussed above. In some embodiments, different voltages and/or currents could be supplied to the cooler 640 to cool the vapor produced by the atomizer 620 and/or to cool one or more other components of the vaporization device 600 to any of various temperatures.

Cooling temperatures could be set by a user input device 656, and/or determined based on such parameters as any one or more of: the type of vaporization substance 603, the condensation temperature of the vaporization substance, the vaporization temperature produced in the atomizer 620, the vapor temperature at one or more measuring or sensing points along a channel, length of a channel, composition of a channel, intake air temperature, and desired vapor output temperature at the mouthpiece 650.

In some implementations, the chamber 602 could include an indicator of its vaporization substance 603. Using this indicator, a controller 654 could determine a condensation temperature for the vaporization substance 603, and control the power delivered to the cooler 640 to maintain vapor temperature near but above the condensation temperature. Alternatively, the condensation temperature of the vaporization substance 603 could be provided by a user using a user input device 656.

Power to the cooler 640 could also or instead be controlled based on a temperature reading by one or more temperature sensors. Incoming vapor temperature in the channel 623 and/or outgoing vapor temperature in the channel 649, for example, could be sensed and used by a controller 654 to turn the cooler 640 on or off, and/or to control a cooling temperature of the cooler 640. Power supplied to the cooler 640 could be turned off, or the cooler could be otherwise disabled, if a sensed vapor temperature is at a desired temperature or within a desired temperature range. In some implementations, a sensor could detect the presence of condensation of vapor in the channel 649, and the controller(s) 654 could control the cooler 640 to decrease cooling when condensation is detected.

Heat transfers from the cooler 640 to the atomizer 620 and/or to the chamber 602 could also or instead be controlled using a controller at 654. In some implementations, one or more temperature sensor(s) are implemented in the chamber 602 and/or the atomizer 620. Sensed temperatures in the chamber 602 and/or the atomizer 620 could be used to determine whether heat transfer from the cooler 640 to these components is viable and/or beneficial. For example, if a sensed temperature of the vaporization substance 603 is below a temperature that is associated with a desired viscosity for the vaporization substance, then heat transfer at 641 could be increased by the controller 654. For example, the controller 654 could control a heat exchanger in the cooler 500 to increase a rate of heat transfer to the chamber 602.

Power and/or control connections for an active cooling element in the cooler 640 could be provided at least in part by a channel. For example, connections could be located inside or outside any or all of the channels 621, 623, 649. In some embodiments, a base, an atomizer, and a stem, or elements therein, act as a conductor to provide a connection that delivers power to an active cooling element from a battery in a battery compartment with which the base engages. However, one or more separate electrical conductors could be provided, for example, from a base and along an inner or outer wall of a stem, along an outer or inner wall of a chamber, and/or elsewhere in a vaporization device to deliver power to an active cooler. An active cooling element could be electrically coupled to power and/or control terminals or connections in an atomizer, with internal conductors inside a stem for example. Conductors could be implemented using transparent conductors, such as indium tin oxide films, so that they are not noticeable to a user. Alternatively, a separate power source such as a battery could be provided to power an active cooling element.

Some components of the vaporization device 600 could be easier to clean, and/or be less affected by residue, than other components. For example, it could be much easier for a user to detach and clean the mouthpiece 650 than the atomizer 620. The cooler 640 could be positioned at or upstream of, or potentially integrated with, such easier cleaned or less affected components. Therefore, if condensation occurs in come components as a result of vapor cooling, the condensation might not significantly affect these components. Some components could also or instead be less prone to inducing condensation, by having smoother surfaces for example. Implementing these components downstream of the cooler 640 could reduce the rate of condensation in the vaporization device 600. Vapor cooling temperatures may also or instead be determined and set to temperatures that are not expected to result in residue buildup within at least a certain distance along a channel or at least at certain components of a vaporization device. Residue buildup might be less problematic in the mouthpiece 650, for example, and vapor cooling temperatures could be set to help prevent or reduce residue buildup at least upstream from the mouthpiece.

A specific example of a vaporization device 600 is shown in FIG. 6. Other embodiments are also contemplated. For example, multiple chambers to store respective vaporization substances could be provided. A chamber could be in fluid communication with a respective atomizer, multiple chambers could supply their respective vaporization substances to the same atomizer, and/or one or more chambers could supply their vaporization substance(s) to a channel or other component and not to an atomizer. Multiple channels, in fluid communication with different atomizers, chambers, or air intakes for example, could be provided.

Either or both of the valve 612 and the vapor valve 622 could be excluded in other vaporization devices. Valves or vapor valves could also or instead be provided in different channels.

More than one cooler 640 could also be provided in some embodiments. The additional cooler(s) could be implemented in fluid communication with the cooler 640, upstream and/or downstream of the cooler 640 in a direction of vapor flow.

Although the channels 621, 623, 649 are all illustrated separately, these channels could instead form a single continuous channel from the atomizer 620 to the mouthpiece 650. At least a portion of the vapor valve 622 and/or the cooler 640 could be inside of this continuous channel.

In an embodiment, a heat sink or even multiple heat sinks, and/or other types of coolers or cooling elements, are removably installed in the channel 649, in the mouthpiece 650, and/or between the channel and the mouthpiece. The heat sink(s), cooler(s), and/or cooling element(s) may be held in place magnetically or otherwise. In some embodiments, a heat sink is removable so that it can be cooled by refrigeration before use.

The vaporization substance 603 could be in the form of a dry substance, liquid, gel and/or a wax, and could have any of various effects. For example, some vaporization substances could include one or more active ingredients that have a psychoactive effect, whereas others could include flavorants such as any one or more of: terpenes, an essential oil, and a volatile plant extract. In a multi-chamber embodiment, one or more vaporization substances could contain an active substance, and others could include flavorants. A user could, using one or more user input devices 656, selectively vaporize the active substance(s) and flavorant(s) to create a controllable mixture of vapors produced from the vaporization substances. This mixture could be tuned for a specific effect, flavor and/or aromatic profile desired the by the user.

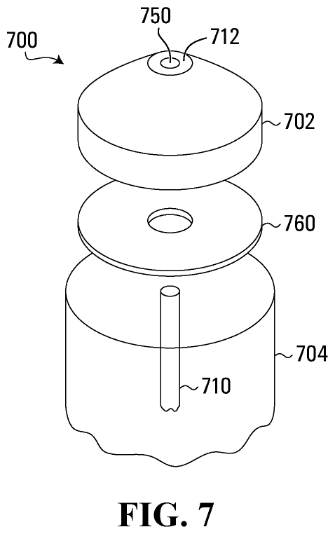

FIG. 6 illustrates an example of a vaporization device with a cooler to cool vapor in a channel that is downstream of an atomizer. Other examples of such a cooler are provided in FIGS. 7 to 18.

FIG. 7 is an isometric and partially exploded view of another example vaporization device 700, which includes a cooler 760. The example vaporization device 700 also includes a cap 702 with a tip 712 and a hole 750 through which a user inhales, and a chamber 704 with a stem 710. The cap 702 could be considered to provide a mouthpiece. The cap 702, the chamber 704, and the stem 710 could be the same as those disclosed in other embodiments herein. The vaporization device 700 could also include other components, such as a base and a battery compartment as in other disclosed embodiments.

The top of the chamber 704 and the stem 710 engage the cap 702 when the vaporization device 700 is assembled. The stem 710 and the hole 750 could be considered to provide a channel that extends through the chamber 704 and the cap 702 to the tip 712.

The vaporization device 700 could carry the cooler 760 in any of various ways. For example, the cooler 760 may be integrated with the cap 702. The cap 702 may be molded around the cooler 760 to encapsulate the cooler. The cooler 760 may also or instead be coupled to the cap 702, by adhesive or otherwise. A friction fit engagement between the cooler 760 and the cap 702 may also or instead be used to couple the cooler to the cap, to have the cooler carried by inside surfaces of outer cap walls, by one or more structures such as posts at a bottom surface of the cap, or otherwise carried in a cavity in the cap, for example.

The cooler 760 need not necessarily be carried by the cap 702, and may instead be coupled to or integrated with the stem 710, to the chamber 704, or to another component. For example, a carrier or adapter could be provided between the cap 702 and the chamber 704 and/or the stem 710 to carry the cooler 760, and the cooler could be coupled to that carrier or adapter.

The cooler 760 may be implemented in any of a number of ways. In some implementations, the cooler 760 is or includes a thermal conductor to conduct heat away from the vapor. By way of example, the cooler 760 could conduct heat from the vapor to the cap 702, where the cap itself could act as a heat sink. The cap 702 could also include one or more thermal conductors, in the form of veins or rings on an outer surface of the cap for example, to conduct heat from the cap to ambient air to help prevent overheating the cap. If the cap 702 provides a mouthpiece, then overheating the cap could result in discomfort for a user, and this can be taken into account in cap design, to divert heat that is absorbed from the vapor downward into the chamber 704 or otherwise away from the tip 712 and/or to provide thermal insulation at least on one or more parts of the cap expected to come into contact with a user's lips during use of the vaporization device 700, for example.

In some implementations, the cooler 760 is or includes a heat sink to absorb heat from the vapor. Such a heat sink may include air or a liquid that is held in a chamber formed by a rigid material, for example. This rigid material is thermally conductive, and may be made from or at least include one or more thermally conductive materials. A phase change material may also or instead be included in such a heat sink.

In some implementations, the cooler 760 is or includes a removable cooling element such as a removable heat sink element, for example. Such a removable heat sink element could be coupled to the cap 702 in the embodiment in FIG. 7, or more generally coupled to an apparatus such as a cartridge or a vaporization device, by a releasable coupling. A friction fit engagement with the cap 702 represents one example of a releasable engagement, and may potentially be applied to a vaporization device component other than a cap. A threaded engagement is another example. A removable heat sink element may also or instead be coupled to a cap or other part of an apparatus such as a cartridge or a vaporization device magnetically.

A removable heat sink element could be removed for cleaning to remove any residue resulting from the condensation of vapor, for example, and then re-installed. A removable heat sink element could also or instead be removed and cooled by refrigeration before use.

The manner by which the cooler 760 receives heat from vapor in the stem 710 and/or the cap 702 is not limited herein. In some implementations, the cooler 760 is in fluid communication with the channel defined by the stem 710 and the cap 702. For example, the cooler 760 could form part of the channel, and thereby directly absorb heat from the vapor to reduce the temperature of the vapor. In some implementations, the cooler 760 could be positioned around, and possibly be in contact with, an outer surface of the stem 710 to indirectly cool the vapor by absorbing heat from the stem. The stem 710 could be made from or at least include a thermally conductive material such as a metal, to improve heat transfer from the vapor to the cooler 760. This thermally conductive material may be considered a form of a passive cooling element.

The location of the cooler 760 along the channel could be determined based on one or more parameters such as expected vapor temperature exiting an atomizer that is in fluid communication with the stem 710, measured vapor temperature exiting the atomizer, expected vapor temperature drop along the stem, measured vapor temperature drop along the stem, and/or vapor condensation temperature, for example. In some implementations, the cooler 760 is positioned proximate to the tip 712 of the cap 702, to limit the length of channel that cooled vapor will traverse before inhalation by a user. This could also limit the length of channel in which condensation of the cooled vapor could occur.

In some implementations, the cooler 760 transfers heat from the vapor to the chamber 104. The cooler 760 could be considered a form of heat exchanger in these implementations. For example, the cooler 760 could include a thermal conductor that is at least partially inside of the chamber 704 and/or that defines an inner surface of the chamber. The thermal conductor could transfer heat from the vapor to the vaporization substance in the chamber 704 to heat the vaporization substance. Transferring heat to the vaporization substance could reduce the viscosity of the vaporization substance, and could reduce the amount of vaporization substance that clings to the surfaces of the chamber 704. This could be particularly beneficial if the vaporization device 700 is stored with the tip 712 facing downwards, and a portion of the vaporization substance in the chamber 704 clings to a surface of the cooler 760 when the vaporization device is reoriented for use. Heat from the vapor that is generated when the vaporization device 700 is in use may be transferred to this portion of the vaporization substance by the cooler 760, which in turn may decrease the viscosity of the vaporization substance and allow the vaporization substance to flow away from the cooler more quickly than if it were not heated. This improved flow may reduce waste, in that the vaporization substance may return to the chamber 704 for vaporization rather than remaining at or near the cooler 760.

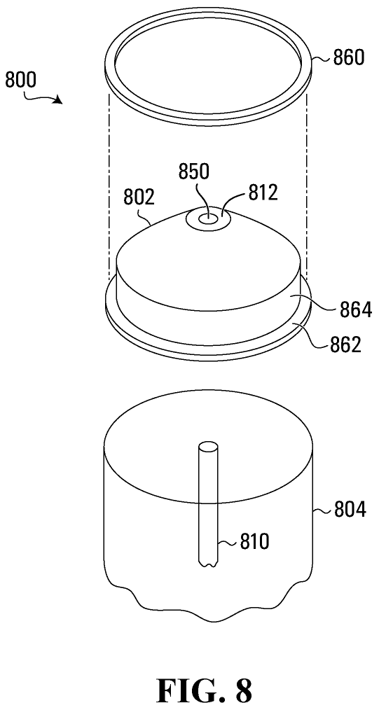

FIG. 8 is an isometric and partially exploded view of a further example vaporization device 800, which includes a cooler in the form of a heat sink 860. The example vaporization device 800 also includes a chamber 804 with a stem 810, and a cap 802 with a flange 862, a side wall 864, a tip 812 and a hole 850 through which a user inhales. The chamber 804 and the stem 810 could be the same as those disclosed in other embodiments herein. The vaporization device 800 could also include other components, such as a base and a battery compartment as in other disclosed embodiments.

The top of the chamber 804 and the stem 810 engage the cap 802 when the vaporization device 800 is assembled. The stem 810 and the hole 850 could be considered to provide a channel that extends through the chamber 804 and the cap 802 to the tip 812.

The heat sink 860 is annular or ring-like in shape. In some implementations, the heat sink 860 is in the form of a nut. Although illustrated as having a rectangular cross-section, the heat sink 860 could instead have a circular or triangular cross-section, for example. The structure and/or material(s) of the heat sink 860 could be similar to those of the cooler 760, or any other heat sink described herein for example. The heat sink 860 is sized and shaped to fit within the space defined by the flange 862 and the side wall 864 of the cap 802. This is illustrated using dashed lines in FIG. 8.

In some implementations, one or more engagements are provided to hold or couple the heat sink 860 to the cap 802. Friction fit engagements and adhesives are examples of such engagements. Optionally, the heat sink 860 is a removable heat sink element that is releasably coupled to the cap 802. A threaded engagement could enable the heat sink 860 to be screwed onto and off of the cap 802. For example, an inner wall of the heat sink 860 could include threads that correspond to threads provided on the side wall 864. The heat sink 860 could also or instead be coupled to the cap 802 magnetically, and a magnetic coupling may be facilitated by a heat sink and cap that are made from or at least include magnetic material(s).

The heat sink 860 is provided to absorb heat from vapor that flows through a channel in the cap 802. In some implementations, the cap 802 includes a thermally conductive material and/or a fluid that conducts heat from vapor to the heat sink 860. The cap 802 could be considered a form of heat exchanger in these implementations.

In the case that the heat sink 860 is releasably engaged with the cap 802, the heat sink may be removed and refrigerated prior to use. The heat sink 860 may also or instead be replaced for another heat sink during use of the vaporization device 800 when the heat sink becomes too warm to provide effective cooling, for example. In some implementations, a heat sink includes an indicator with a temperature sensitive material, such as a thermochromic ink for example, that indicates when the heat sink is too warm to provide effective cooling and should be replaced.

Although only one heat sink is shown in each of FIGS. 7 and 8, multiple heat sink elements may be provided for a higher cooling capacity. Multiple heat sink elements may be releasably coupled to each other, and/or to an apparatus, magnetically or otherwise. In an embodiment, a vaporization device includes both of the heat sinks 760, 860.

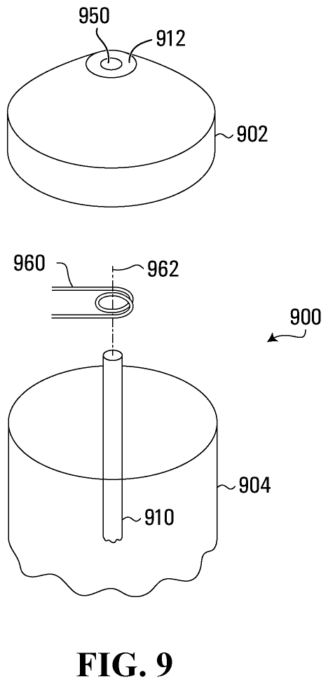

FIG. 9 is an isometric and partially exploded view of yet another example vaporization device 900. The vaporization device includes a chamber 904 with a stem 910 and a cooler 960, and a cap 902 with a tip 912 and a hole 950 through which a user inhales. The cap 902, the chamber 904, and the stem 910 may be similar to those disclosed elsewhere herein. The vaporization device 900 may also include other components, such as a base and a battery compartment as in other disclosed embodiments.

The top of the chamber 904 and the stem 910 engage the cap 902 when the vaporization device 900 is assembled. The stem 910 and hole 950 may be considered to provide a channel that extends through the chamber 904 and the cap 902 to the tip 912.

The cooler 960 includes an element with a turn that, when installed, surrounds and could be in contact with an outer wall of the stem 910. This is represented in FIG. 9 by the dashed line 962. The location of the cooler 960 along the stem 910 could be determined based one or more parameters such as expected vapor temperature exiting an atomizer that is in fluid communication with the channel, measured vapor temperature exiting the atomizer, expected vapor temperature drop along the stem 910, measured vapor temperature drop along the stem, and/or vapor condensation temperature, for example.

The chamber 904 and/or another part of a vaporization apparatus such as a cartridge or a vaporization device may carry the cooler 960 in any of various ways. For example, the cooler 960 may be integrated with the stem 910 or the cap 902. The cap 902 may be molded around the cooler 960 to encapsulate at least part of the cooler. The cooler 960 may also or instead be coupled to the cap 902 and/or another part of an apparatus, by adhesive or otherwise. A friction fit engagement between the cooler 960 and part of an apparatus may also or instead be used to couple the cooler to an apparatus. In some embodiments, the cooler 960 is or includes a removable cooling element, and is coupled to an apparatus by a releasable coupling, examples of which are disclosed elsewhere herein.

The cooler 960 may be solid or hollow, and formed from or at least include a thermally conductive material such as a metal. In some implementations, the cooler 960 is or includes a heat exchanger, to transfer heat away from the vapor and/or the stem 910. This heat may be transferred to a heat sink, the chamber 904, an atomizer, and/or the ambient atmosphere, for example. The cooler 960 may also include a heat sink, which is or includes a material inside the heat exchanger. For example, the cooler 960 may be hollow, with a heat sink in the form of a fluid inside. The fluid may be a gas such as air or a liquid such as a refrigerant. The gas or liquid may be circulated by an active cooling element, such as a fan or pump for example, to transfer heat away from the stem 910. In the case of air as the heat sink, outside air may be circulated through the cooler 960. A heat sink material may instead be part of a closed, sealed system.

In the examples of a gas or liquid heat sink, the heat sink is inside the cooler 960 and is thermally coupled to the vapor, indirectly through the stem 910. In other embodiments, a heat sink is physically in contact with or otherwise thermally coupled to a heat exchanger without being inside the heat exchanger.

As noted elsewhere herein, a cooler may be or include one or more passive cooling elements and/or one or more active cooling elements. Any or all active cooling elements in the cooler 960 may be coupled to a power source and/or a controller, which may be provided in a battery compartment and/or a base of a vaporization device, for example. Power and/or control connections for an active cooling element may be located inside or outside a vaporization device channel. In some embodiments, a base, an atomizer, and a stem such as 910, and/or elements therein, act as a conductor to provide a connection that delivers power to one or more active cooling elements from a battery in a battery compartment with which the base engages. However, one or more separate electrical conductors could be provided, for example, from a base and along an inner or outer wall of a stem such as 910, along an outer or inner wall of a chamber such as 904, and/or elsewhere in a vaporization device to deliver power to an active cooling element. An active cooling element may be electrically coupled to power and/or control terminals or connections in an atomizer, with internal conductors inside a stem such as 910 for example. As noted at least above, conductors may be implemented using transparent conductors, such as indium tin oxide films, so that they are not noticeable to a user. Alternatively, a separate power source such as a battery may be provided to power an active cooling element.

FIG. 9 illustrates an example of a cooler 960 that is outside a channel, in particular outside the stem 910. In some embodiments, a cooler is at least partially inside a channel. For example, a cooling element such as the cooler 960, or at least the turn or coil in the example shown, may be inside the stem 910.

The cooler 960 is in the shape of a coil, which is an example of a surface area increasing structure to increase a surface area for heat transfer. A surface area increasing structure need not be in the form of a coil, and could take other shapes or forms. Non-limiting examples of other surface area increasing structures include one or more bumps, protrusions, grooves, flanges, ribs, ridges, meshes, grids, plates, rings, and sleeves. In some embodiments, a surface area increasing structure includes a rough or roughened surface. A surface area increasing structure could be implemented inside or outside a vaporization device channel, or form part of a channel. A surface area increasing structure could be formed during fabrication of a component of a vaporization apparatus, or could be formed by roughening a substantially smooth surface of a vaporization apparatus through machining or etching, for example.

Alternatively, a jacket or sleeve that includes a rough surface could be coupled to a channel or other component of a vaporization apparatus.

Although the cooler 960 is shown in FIG. 9 with a cooling coil that has only one turn, a cooler could have multiple turns to provide a higher cooling capacity. Multiple separate coils could also or instead be provided.

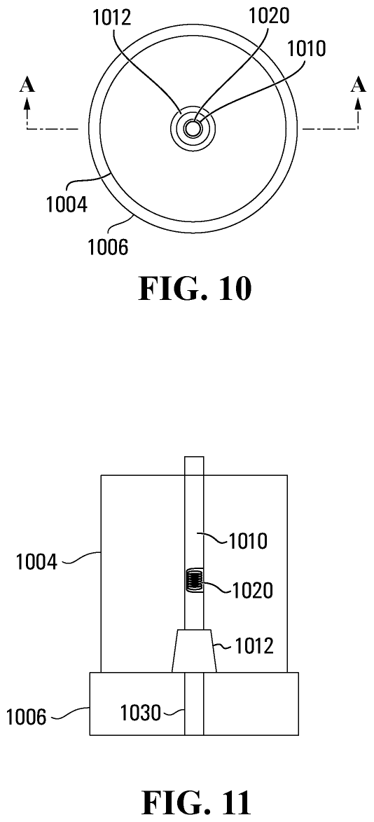

FIG. 10 is a top view of an example chamber 1004 with a cooler 1020, and FIG. 11 is a cross-sectional view of the chamber 1004, along the line A-A in FIG. 10. Features referenced below are shown in one or both of FIGS. 10 and 11.

The chamber 1004 engages a base 1006. In a vaporization device, the top of the chamber 1004 and a stem 1010 may engage a cap, and the bottom of the base 1006 may engage a battery compartment. The stem 1010 provides a channel that is in fluid communication with a channel 1030 in the base in the example shown. The chamber 1004, the base 1006, the stem 1010, and the atomizer 1012 may be similar to the chamber 104, the base 106, the stem 110, and the atomizer 130 discussed above with reference to FIGS. 1 and 2, for example.

The cooler 1020 is illustrated as being located in at position inside the stem 1010. Thus, the cooler 1020 is in fluid communication with the channel provided by the stem 1010, to directly cool vapor in the stem. The location of the cooler 1020 along the stem 1010 may be determined based one or more parameters such as expected vapor temperature exiting the atomizer 1012, measured vapor temperature exiting the atomizer, expected vapor temperature drop along the stem 1010, measured vapor temperature drop along the stem, and/or vapor condensation temperature, for example.

In some implementations, the cooler 1020 is or includes a passive cooling element. For example, the cooler 1020 may be made from a thermally conductive material to transfer heat away from the vapor. The heat could be transferred to the walls of the stem 1010, and possibly to the chamber 1004 to heat and reduce the viscosity of a vaporization substance, for example. Thus, in some implementations, the cooler 1020 may be considered a heat exchanger to transfer heat to the chamber 1004. Heat may also or instead be conducted to a heat sink (not shown). In some implementations, the cooler 1020 is or includes a heat sink material.

The cooler 1020 is the shape of a coil, which is an example of a surface area increasing structure to increase a surface area for heat transfer. More or fewer turns than shown may be implemented in the coil to adjust the amount of cooling provided by the coil 1020. Examples of other types of surface area increasing structures are provided at least above, and may be implemented in other embodiments of a cooler inside a channel.

In some implementations, the cooler 1020 is or includes an active cooling element. For example, the cooler 1020 may include a hollow tube with a pump that circulates a fluid. The fluid may receive heat from vapor in the stem 1010, and transfer the heat to the chamber 1004, to the atomizer 1012, to a heat sink, or to the ambient atmosphere outside the chamber 1004 through additional channels and/or thermal couplings (not shown).

Power and/or control connections may be located inside or outside a channel. In some embodiments, the base 1006, the atomizer 1012, and the stem 1010, and/or elements therein, act as a conductor to provide a connection that delivers power to the cooler 1020 from a battery in a battery compartment with which the base 1006 engages. However, one or more separate electrical conductors could be provided, for example, from the base 1006 and along an inner or outer wall of the stem 1010, along an outer or inner wall of the chamber 1004, and/or elsewhere in a vaporization device to deliver power to the cooler 1020. The cooler 1020 may be electrically coupled to the atomizer 1012 or to power and/or control terminals or connections in the atomizer, with internal conductors inside the stem 1010 for example. Alternatively, a separate power source such as a battery may be provided to power the cooler 1020.

Although the cooler 1020 is shown in FIGS. 10 and 11 as being inside the stem 1010, a cooler may also or instead be implemented at a position inside other components of a vaporization device. For example, a cooler may be implemented inside a channel provided in a cap or mouthpiece, instead of or in combination with the cooler 1020. A cooler may instead be implemented outside a channel so as to avoid restricting flow through the channel and/or clogging of the channel due to condensation as a result of direct cooling of a fluid flow. A cooler may also or instead be positioned away from an area at which the stem 1010 is in contact with the vaporization substance, to the extent that the cooler may also cool the stem and potentially cause viscosity of the vaporization substance to increase and hinder its flow to the atomizer 1012.

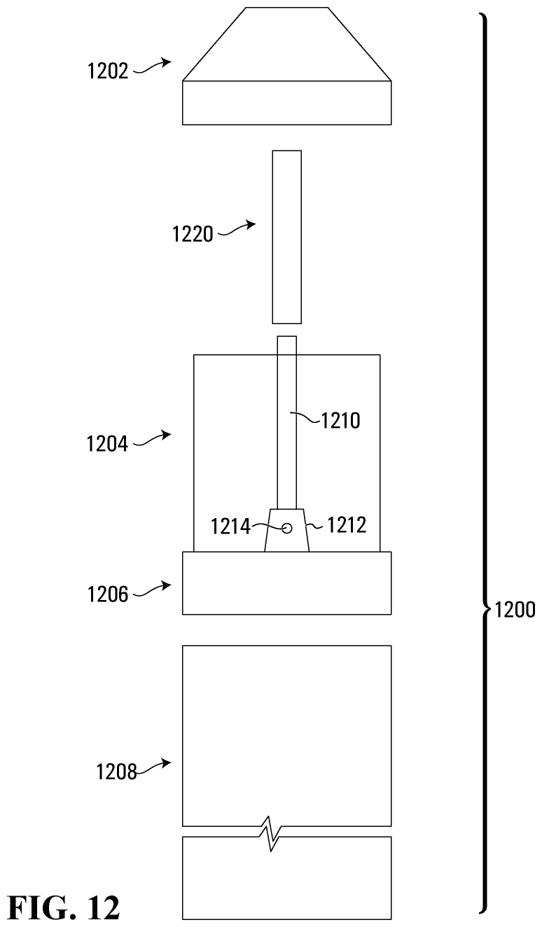

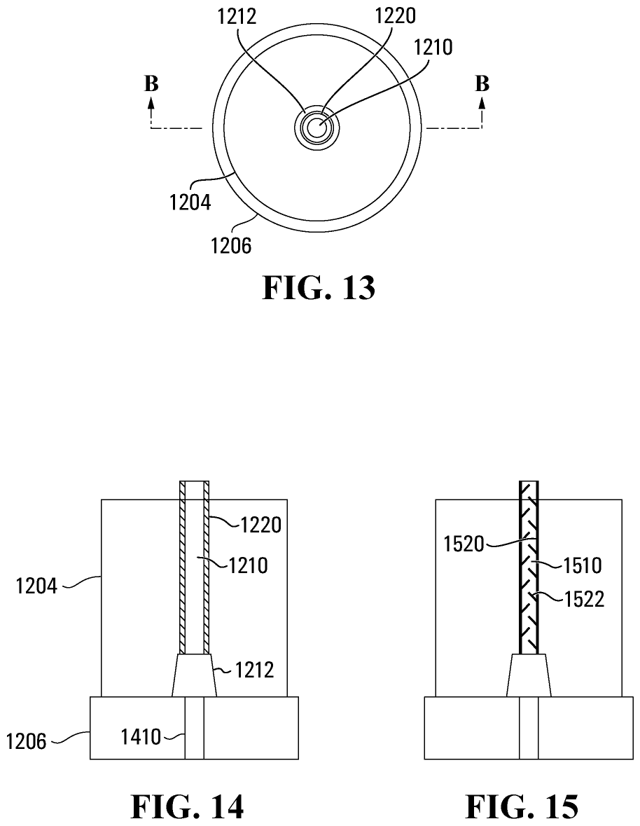

FIG. 12 is a plan and partially exploded view of another example vaporization device 1200, FIG. 13 is a top view of a chamber 1204 in FIG. 12, and FIG. 14 is a cross-sectional view of the chamber in FIG. 13, along the line B-B in FIG. 13. Various features referenced in the description below are shown in one or more of these drawings.

The vaporization device 1200 includes, in part, a cap 1202, a chamber 1204, a base 1206, and a battery compartment 1208. A stem 1210, an atomizer 1212, and an intake hole 1214 are also shown inside the chamber 1204. These components could be similar to the cap 102, the chamber 104, the base 106, the battery compartment 108, the stem 110, the atomizer 130, and the intake hole 134 discussed above with reference to FIGS. 1 and 2, for example. A cooler 1220 is also shown, and in this embodiment the cooler is to cool the stem 1210 along at least part of its length.

The cooler 1220 is a tube or sleeve that fits over the stem 1210. When assembled, the cooler 1220 covers at least part of the stem 1210, and the top ends of at least the chamber 1204 and the stem 1210 engage the cap 1202. The chamber 1204 and base 1206 are illustrated in an assembled state, with the chamber engaging the base. The base 1206 also engages the battery compartment 1208 when the device 1200 is fully assembled. Examples of cap/chamber/stem/base/battery compartment engagements are described elsewhere herein, at least with reference to FIGS. 1 and 2.

The cooler 1220 may, but need not necessarily in every embodiment, be sealed against the stem 1210, the atomizer 1212, and/or the cap 1202. Sealing elements such as O-rings or gaskets may be used to provide seals.

The cooler 1220 may be sealed from a vaporization substance in the chamber 1204. For example, the cooler 1220 may be enclosed within a material or otherwise sealed from the vaporization substance so that the cooler can be located at an outside part of the stem 1210, as shown perhaps most clearly in FIGS. 13 and 14, without contacting the vaporization substance. Alternatively, the cooler 1220 may be at least partially in contact with the vaporization substance in the chamber 1204 to transfer heat to the vaporization substance, for example.

The atomizer 1212 is in fluid communication with the chamber 1204, through the intake hole 1214, to generate vapor from the vaporization substance by heating the vaporization substance. An air intake hole or passage 1410 (FIG. 14), which is provided in the base 1206, is in fluid communication with the atomizer 1212 to carry air to the atomizer. The stem 1210 provides a channel in fluid communication with the atomizer 1212, to carry air and vapor away from the atomizer. The cap 1202, which may be or include a mouthpiece, is also in fluid communication with the channel in the stem 1210.

According to the embodiment shown in FIG. 14, the stem 1210 and the vapor in the stem are thermally cooled by the cooler 1220 along its entire length. More generally, a cooler may extend at least partially along the stem 1210. The entire stem 1210 need not necessarily be cooled. Characteristics such as the type(s) of cooler and/or how much of a channel is cooled may be determined based on any one of more of various parameters. Examples of such parameters include user input, expected vapor temperature exiting the atomizer 1212, measured vapor temperature exiting the atomizer, expected vapor temperature drop along the stem 1210, measured vapor temperature drop along the stem, and/or vapor condensation temperature.

Vapor in the stem 1210 may be indirectly cooled by the cooler 1220. For example, heat from the vapor may be conducted through the stem 1210 to the cooler 1220. The stem 1210 may be at least partially made from a thermal conductor to increase the rate of heat transfer to the cooler 1220. The cooler 1220 may also or instead be in fluid communication with the channel defined by the stem 1210, by being at least partially located at a position inside the channel for example, to directly cool the vapor.

The cooler 1220 may be or include a heat exchanger to transfer heat from the stem 1210 to the chamber 1204. This may heat the vaporization substance in the chamber 1204 to reduce the viscosity of the vaporization substance and inhibit the vaporization substance from clinging to the cooler 1220 or the stem 1210, for example.

In some implementations, the cooler 1220 includes one or more active cooling elements. For example, a thermoelectric cooling element may be implemented with a cool side facing towards the stem 1210, and a hot side facing toward the chamber 1204. The thermoelectric cooling element may be powered by connections in the engagement between the cooler 1220 and the atomizer 1212, for example. During use, the cool side of the thermoelectric cooling element cools the vapor flowing through the channel in the stem 1210. The hot side of the thermoelectric cooling element may heat the chamber 1204 and any vaporization substance held therein. The thermoelectric cooling element may be in direct contact with the vaporization substance. Alternatively, a thermal conductor may encapsulate the thermoelectric cooling element, to transfer heat from the thermoelectric cooling element to the chamber 1204.

Although illustrated as being cylindrical in shape, the stem 1210 may instead have another shape, such as rectangular or triangular. These other example shapes provide flat surfaces, on which it may be easier to implement a thermoelectric cooling element.

In some implementations, the cooler 1220 is or includes a heat sink. For example, the cooler 1220 may define a hollow cavity holding a gas or liquid to absorb heat from the stem 1210. Active cooling elements such as fans and pumps may be provided to circulate the gas or liquid through the cooler 1220, to potentially increase the rate of heat absorption. A hollow cavity in the cooler 1220 may also or instead include a phase change material as a heat sink.

FIGS. 12 to 14 illustrate an embodiment in which a cooler 1220 is located at an outside part of a stem 1210. Other embodiments are also contemplated. FIG. 15, for example, is a cross-sectional view of another example chamber in which a cooler 1520 is located at a position inside a channel. This channel is defined by a stem 1510.

On a comparison of FIGS. 14 and 15, it will be seen that the external cooler 1220 in FIG. 14 is illustrated as being thicker than the internal cooler 1520 in FIG. 15. This is just an example. A thinner cooler 1520 may be preferred as an internal cooler so as to avoid overly restricting the size of the channel through the stem 1210. Alternatively, channel size may be maintained by using a thicker internal cooler with a larger diameter stem.

In some implementations, the cooler 1520 is or includes a passive cooling element. For example, the cooler 1520 may include a thermal conductor to transfer heat away from the vapor. Similar to the cooler 1220, this heat may then be transferred to a chamber and/or to a vaporization substance, for example.

In some implementations, the cooler 1520 includes one or more thermal conductors such as copper, integrated into the walls of the stem 1510 to conduct heat away from the vapor. Although forming the entire stem 1510 from a thermal conductor may provide a relatively high thermal conductivity, this might not be preferable from a cost perspective. Therefore, in some implementations the stem 1510 includes lines, bands, or veins of a thermal conductor to conduct heat away from the vapor. For example, veins may be provided in the form of rings extending through the stem 1510, lines extending along the axial length of the stem and radially through the stem, and/or other shapes. A stem that includes a thermal conductor is also referred to herein as a heat conductive stem. Veins of a thermal conductor may also help direct the transfer of heat and in that sense also be considered as a form of heat exchanger.

The cooler 1520 includes multiple ribs or fins 1522 that project radially inwardly from the stem 1510. The fins 1522 may extend through the stem 1510 and/or be thermally coupled to one or more veins of a thermal conductor in the stem. The fins 1522 are an example of a surface area increasing structure to increase a surface area for heat transfer. The fins 1522 are at least partially made from a thermally conductive material in some implementations.

The fins 1522 are illustrated as being angled or tilted upwards, in the direction of vapor flow in the stem 1510. This may be useful in reducing drag on the vapor caused by the fins 1522. However, this is only an example. In other embodiments, fins may be perpendicular to an inner wall of the stem, or slanted or tilted against the direction of vapor flow. A cooler having fins with different angles of tilt is also contemplated.

The fins 1522 may be fabricated integrally with the stem 1510, or coupled to the stem 1510 using adhesives or fasteners, for example. In some implementations, each of the fins 1522 is a discrete rod or plate that projects from the inner wall of the stem 1510. The fins 1522 may be staggered along the length of the stem 1510 to potentially improve vapor contact with the fins.

The fins 1522 provide additional surface area that contributes to transferring heat away from the vapor. The fins 1522 may also induce turbulent flow, for example, to encourage mixing of the vapor in the channel, and/or potentially increase heat transfer to the cooler 1520.

It should be noted that although the fins 1522 are illustrated inside the stem 1510, fins or any other surface area increasing structure may also or instead be provided in other components of a vaporization device. For example, fins may be provided on an outside surface of a stem to improve heat transfer to a chamber. Fins may also or instead be implemented in a cap, mouthpiece, air intake channel, or atomizer, for example.

Fins are an example of a surface area increasing structure. Others are also possible. In another embodiment, a single spiral element is spiraled along a length of the stem 1510 for example. In some embodiments, it may be preferred to implement vapor cooling without fins projecting into the stem 1510 or other channel so as to avoid restricting flow through the channel and/or clogging of the channel due to condensation.

The coolers 1220 and 1520 are two examples of coolers that can be coupled to or part of a stem. Other examples are also contemplated. In some embodiments, a cooler includes a thermal conductor in the form of a coating on the inner and/or outer surface of a stem.

Coolers may be implemented in other components of a vaporization device to cool a vapor. For example, a cooler similar to either of the coolers 1220, 1520 may be implemented in a cap or mouthpiece to cool vapor that flows in a channel defined by the cap/mouthpiece. The cooler may be in fluid communication with the channel of the cap/mouthpiece, and optionally be located at a position inside the channel. A cooler may also or instead be implemented outside a channel so as to avoid restricting flow through the channel and/or clogging of the channel due to condensation as a result of direct cooling of a fluid flow. A cooler may also or instead be positioned away from an area at which a stem or channel is in contact with a vaporization substance, to the extent that the cooler may also cool the stem or channel and potentially cause viscosity of the vaporization substance to increase and hinder its flow to an atomizer.

In some embodiments, a mouthpiece includes a cooler that transfers heat from vapor carried by the mouthpiece to the mouthpiece itself. The mouthpiece then acts as a heat sink to absorb the heat. Heat exchangers may also be implemented in the mouthpiece to transfer heat from the mouthpiece to the ambient air. This may help prevent the mouthpiece from becoming to becoming too warm and causing discomfort to a user or even potentially burning the user.

Whether implemented inside or outside of a stem, cap, mouthpiece or other component of a vaporization apparatus, a cooler may be removable, for replacement or cleaning. For example, a cooler could be placed over or inside a stem without being fastened to the stem, and then slid on or into, and off or out of, the stem. Any fasteners may be released or broken, and then re-fastened or replaced, when a cooler is removed and re-installed or replaced.

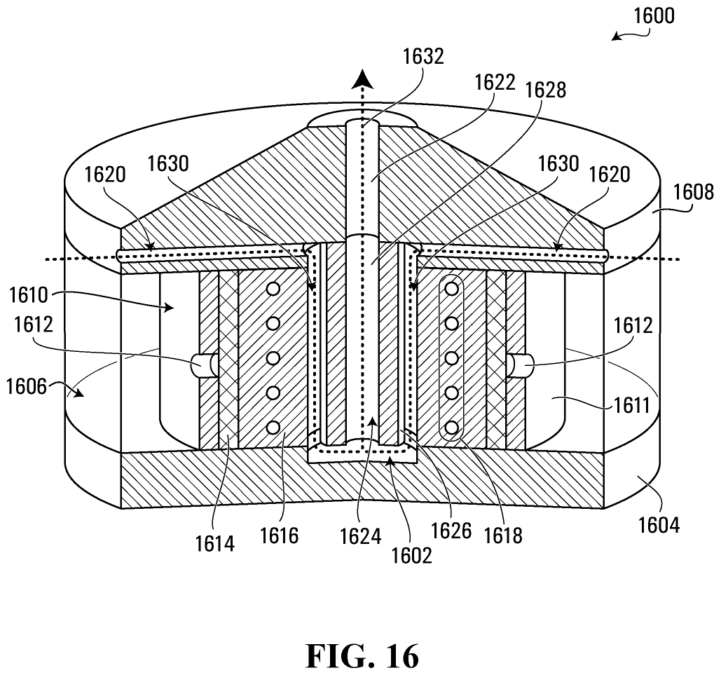

FIG. 16 is a diagram illustrating internal structure of an example vaporization device cartridge 1600 with a cooler 1602. The example cartridge 1600 is shown with a section removed so that internals of the cartridge can be seen. The cartridge 1600 may be implemented in a vaporization device, non-limiting examples of which are provided elsewhere herein.

The cartridge 1600 includes a base 1604, a chamber 1606, and a cap 1608. The base 1604 is engaged with a bottom of the chamber 1606, and the cap 1608 in engaged with a top of the chamber. Example engagements between a chamber, a cap and a base are provided elsewhere herein. The bottom of the base 1604 may also engage with a battery compartment of a vaporization device, for example.

The cap 1608 defines multiple air intake channels 1620 and another channel 1622. In some implementations, the cap 1608 includes or provides a mouthpiece.

Inside the chamber 1606 is an atomizer 1610 that includes an outer wall 1611 having multiple intake holes 1612 therein, a ceramic core 1616 with a heating element 1618, and a wick 1614 disposed between the outer wall and the ceramic core. The wick 1614, the ceramic core 1616, and the heating element 1618 may be similar to the wick 403, the ceramic core 402, and the heating element 404 of FIG. 4, for example.

The atomizer 1610 is a hollow cylinder that defines a chamber or cavity 1630. The cooler 1602 is disposed inside of the cavity 1630. The cooler 1602 is also a hollow cylinder having an outer surface 1626 facing towards the ceramic core 1616, and an inner surface 1628 that defines a channel 1624. The cooler 1602 is arranged or positioned relative to the cap 1608 such that the channel 1624 at least partially aligns with the channel 1622. The channels 1622, 1624 may be considered as forming a single channel. The cooler 1602 may be coupled to the cap 1608 using fasteners and/or adhesives, for example. The cooler 1602 does not extend the full length of the cavity 1630, in order to provide a gap between the cooler and the bottom of the cavity in the example shown. The gap enables fluid communication between the cavity 1630 and the channels 1622, 1624. In another embodiment, the cooler 1602 extends to the bottom of the cavity 1630 and may be coupled to the base 1604, and one or more passages are provided to enable fluid communication between the cavity 1630 and the channels 1622, 1624.

During use, a vaporization substance that is held in the chamber 1606 enters the atomizer 1610 through the intake holes 1612. The vaporization substance can then flow or seep through the wick 1614 and into the ceramic core 1616. Heat produced by the heating element 1618 can heat and vaporize the vaporization substance to produce a vapor. This vapor can then enter the cavity 1630.

When a user draws from a vaporization device that includes the cartridge 1600, outside air is drawn through the air intake holes 1620 and into the cavity 1630. The air then flows through the cavity 1630, between the ceramic core 1616 and the outer surface 1626 of the cooler 1602. In the illustrated example, the air flows in the downwards direction between the ceramic core 1616 and the outer surface 1626 of the cooler 1602. This air mixes with the vapor generated by the ceramic core 1616, and a mixture of air and vapor then flows through the channels 1622, 1624 for inhalation by a user, for example. The general flow of vapor and air in the cartridge 1600 is illustrated by the dashed lines shown at 1632.

In some implementations, the cooler 1602 includes a thermoelectric cooling element. Examples of power and control connections for the thermoelectric cooling element are provided elsewhere herein. The inner surface 1628 may be or include a cold side of such a thermoelectric cooling element, to cool vapor as it flows through the channel 1624.

The outer surface 1626 may be or include a hot side of the thermoelectric cooling element. As such, heat may be transferred from the outer surface 1626 to the cavity 1630 and to the ceramic core 1616. Any or all air, vapor, and vaporization substance in the cavity 1630 may receive heat from the outer surface 1626. If vaporization substance flows through the ceramic core 1616 and reaches the cavity 1630 without being vaporized, heat from the outer surface 1626 may vaporize this vaporization substance, to potentially help provide complete vaporization of a vaporization substance, and inhibit leakage of liquid vaporization substance from the atomizer 1610.

The length of the cooler 1602 in the channel 1624 is implementation specific. The cooler 1602 may cool vapor along the entire length of the channel 1624, or only a portion of the length of the channel. In some embodiments, the cooler 1602 extends into the channel 1622 to cool vapor in the cap 1608.

In some implementations, control of the atomizer 1610 and the cooler 1602 is integrated. For example, if a controller determines that cooler vapor is desired, then power delivered to an active cooling element such as a thermoelectric cooling element in the cooler 1602 may be increased to increase cooling in the channel 1624. This may also increase heating in the cavity 1630 by the outer surface 1626, and may lead to overheating and even combustion of a vaporization substance. As such, the controller may potentially decrease the power delivered to the heating element 1618 when power to the cooler 1602 is increased. This may provide a substantially stable vaporization temperature in the atomizer 1610 while still allowing for variation of cooling temperatures in the channel 1624.

A cooler or cooling element that transfers heat from one component or location in a vaporization device to another component or location may be considered to be serving at least a dual-purpose. Considering a thermoelectric cooling element according to an example above, such an element may be a dual-purpose element to perform heating to generate a vapor, and cooling to cool the vapor. Such a thermoelectric cooling element may also or instead be considered a form of heat exchanger, at least because the thermoelectric cooling element transfers heat from the vapor in the channel 1624 to the atomizer 1610.

Heat transfer from a vapor stream to a chamber and/or a vaporization substance in a chamber is another example of a dual-purpose cooling/heating feature. Multi-purpose features such as the dual-purpose features noted by way of example above are not limited only to active coolers or cooling elements, and may also or instead be provided by passive coolers or cooling elements.

The cartridge 1600 is provided by way of example, and other implementations are also contemplated. For example, a cartridge and/or a cooler might not be cylindrical in shape, and could instead be rectangular or triangular in shape. It should also be noted that the atomizer 1610 and/or elements thereof need not extend all the way to the cap 1608 in other embodiments. A stem, for example, may extend from the atomizer to the cap 1608 and engage the bottom of the cap, such that the air intake holes 1620 are in fluid communication with an interior of the stem and the cooler 1602 also extends into the stem.

Other features may also or instead be provided. For example, to the extent that condensation may potentially build up at the base of the cooler 1602 and obstruct fluid flow, such a feature as pre-heating of the base by the heating element 1618 and/or a separate heating element when a vaporization device is turned on may be useful in melting away excess vaporization substance or condensation and thereby reducing or preventing clogging.

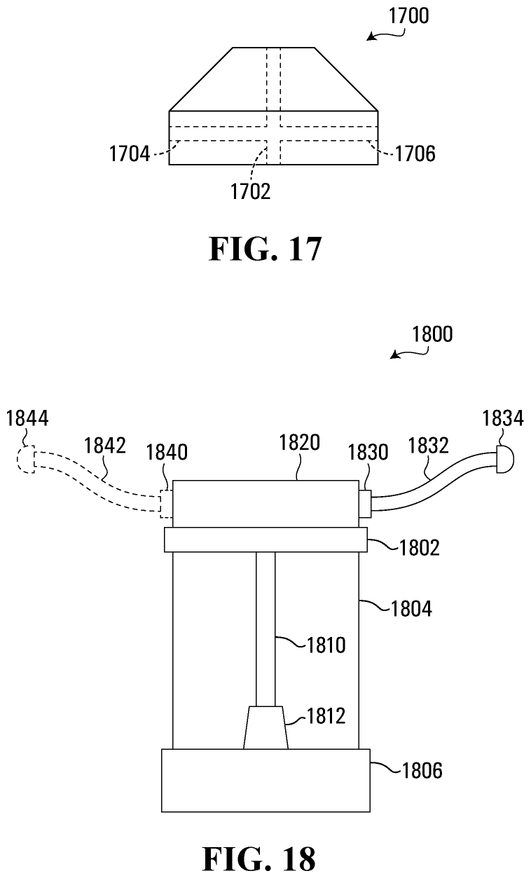

In some embodiments, vapor is cooled by mixing the vapor with ambient or outside air. FIG. 17 is a plan view of an example cap 1700, with a channel 1702 for fluid communication with a mouthpiece, which may be part of the cap or a separate component. The cap 1700 includes additional air intake channels 1704, 1706 in fluid communication with the channel 1702. The additional air intake channels 1704, 1706 include openings to draw ambient air into the cap 1700.

Cooling by intake air as shown by way of example in FIG. 17 may be implemented in a cap 1700 and/or in a mouthpiece that is in fluid communication with a channel through which vapor flows. In some implementations, a cap and/or a mouthpiece is coupled to an apparatus such as a chamber, a cartridge, or a vaporization device that includes an atomizer to generate a vapor, and a channel to carry the vapor to the channel 1702. A cap or a mouthpiece may be coupled an apparatus by a threaded engagement, by a friction fit engagement, and/or by some other type of releasable engagement, for example. A cap or a mouthpiece may instead be coupled to the apparatus using a non-releasable engagement. In general, for cooling that is to be provided by intake air, intake air or additional intake air may be allowed into a mouthpiece, into a channel through which vapor is provided to a mouthpiece, and/or into one or more parts of a vaporization device channel upstream from a channel through which vapor is provided to a mouthpiece.

When the cap 1700 is coupled to an apparatus, the air intake channels 1704, 1706 may be coupled to the channel 1702 at a position downstream of an atomizer in a direction of vapor flow. During use of the apparatus, ambient air from outside the cap 1700 can enter the cap through the channels 1204, 1206 and mix with vapor in the channel 1702 to cool the vapor. As such, the air intake channels 1704, 1706 may be considered to be further examples of coolers or cooling elements.

Control of intake air flow in the channels 1704, 1706 may be manual and/or automatic. In some implementations, the cap 1700 includes one or more valves and/or other air flow control components, also referred to herein generally as regulators, to control the flow of air through either or both of the air intake channels 1704, 1706. In some embodiments, these regulators are controllable and powered active cooling elements.

As an example of manual control, a user may manually control intake air flow by operating one or more valves and/or other user input devices to provide a desired temperature at an outlet of the channel 1702. A user may also or instead cover or partially cover an inlet of one or more air intake channels 1704, 1706, using a finger, thumb, and/or lip for example. A structure such as a rotatable perforated ring or band around a lower part of the cap 1700 in the view shown in FIG. 17 may be provided for operation by a user to control the extent to which one or more air intake channel inlets are open to admit air flow into the channel 1702. Other types of sliding, rotating, or otherwise movable inlet covers, including respective separate covers for one or more inlets and one or more covers to control air flow through multiple inlets, are possible.

Automatic control may be responsive to one or more temperature sensors to sense the temperature of air in a channel, such as the channel 1702 and/or an upstream channel in fluid communication with the channel 1702, and provide measurements and/or other signals to control operation of one or more air flow control components. Another intake air control option is to control one or more air flow control components based on operation of an atomizer. For example, an atomizer and one or more intake air flow control components may be operated or controlled together, to increase intake air flow when the atomizer is operating at higher temperatures and to decrease intake air flow when the atomizer is operating at lower temperatures. Cooler operation and/or control may also or instead be associated with operation and/or control of other components.

FIG. 17 illustrates an example of an embodiment in which a cap or mouthpiece may provide a cooling effect. Vapor cooling could also or instead be provided by implementing a longer channel for vapor to travel through, to provide time for vapor to cool before reaching a mouthpiece. FIG. 18 is a plan view of another example cartridge 1800 that includes such longer channels.

The example cartridge 1800 includes a chamber 1804 with a stem 1810 and an atomizer 1812, and a base 1806 engaged with the chamber 1804. These components may be similar to those disclosed by way of example elsewhere herein. A cap 1802 engages the chamber 1804 and the stem 1810, and the engagements of the chamber and the stem with the cap may also be as disclosed elsewhere herein. A mouthpiece 1834 is coupled to the cap through a hose or pipe 1832, a connector 1830, and a manifold 1820 in the example shown, but in other embodiments the hose may be coupled to the cap 1802. Multiple mouthpieces may be provided in some embodiments, and a second mouthpiece 1844, hose 1842, and connector 1840 are shown in FIG. 18.

The manifold 1820 provides multiple channels in fluid communication with a channel through the stem 1810, and may be made from the same material(s) as the cap 1802 and/or different material(s). The manifold 1820 and the cap 1802 may be integrated together into a single component in some embodiments.

The connectors 1830, 1840 may be, for example, threaded connectors to couple the hoses 1832, 1842 to the manifold 1820. Any of various types of connectors, made from the same material(s) as the manifold 1820 and/or different material(s), maybe used for this purpose. Threaded connectors, friction fit connectors, magnetic connectors and/or other types of connectors may be used. The manifold 1820 and/or the connectors 1830, 1840 may include valves or other regulators that open channels through the connectors only when a mouthpiece hose 1832, 1842 is connected.

The hoses 1832, 1842 may be made from any of various materials, such as rubber or plastic. Hoses 1832, 1842 that are made from, or at least include, a thermally conductive material, may improve vapor cooling as vapor travels along a hose. For example, the hoses 1832, 1842 may be made from or at least include materials with a high thermal conductivity, such as copper, to help cool the vapor. Each hose 1832, 1842 may include an adapter or other structure to engage the connectors 1830, 1840.

Examples of materials from which mouthpieces 1834, 1844 may be made are disclosed elsewhere herein. The mouthpieces 1834, 1844 may be integrated with the hoses 1832, 1842 or attached to the hoses. Threaded engagements, friction fit engagements, magnetic engagements and/or other types of engagements may be used.

In FIG. 18, the mouthpiece 1834 is in fluid communication with the channel provided by the stem 1810, through a further channel that is provided by the hose 1832 and the manifold 1820. In an embodiment with multiple mouthpieces, the mouthpieces 1834, 1844 are in fluid communication with the channel provided by the stem 1810, through respective further channels that are provided by the hoses 1832, 1842 and the manifold 1820.

In some implementations, the manifold 1820, the hose 1832 and/or the mouthpiece 1834 include a thermal conductor to transfer heat away from the vapor. The thermal conductor may be in the form of veins or rings of metal, for example. Other forms of thermal conductor and/or other vapor cooling features, such as those described at least above in the context of heat conductive stems, may also or instead be provided in or by a hose 1832, 1842.

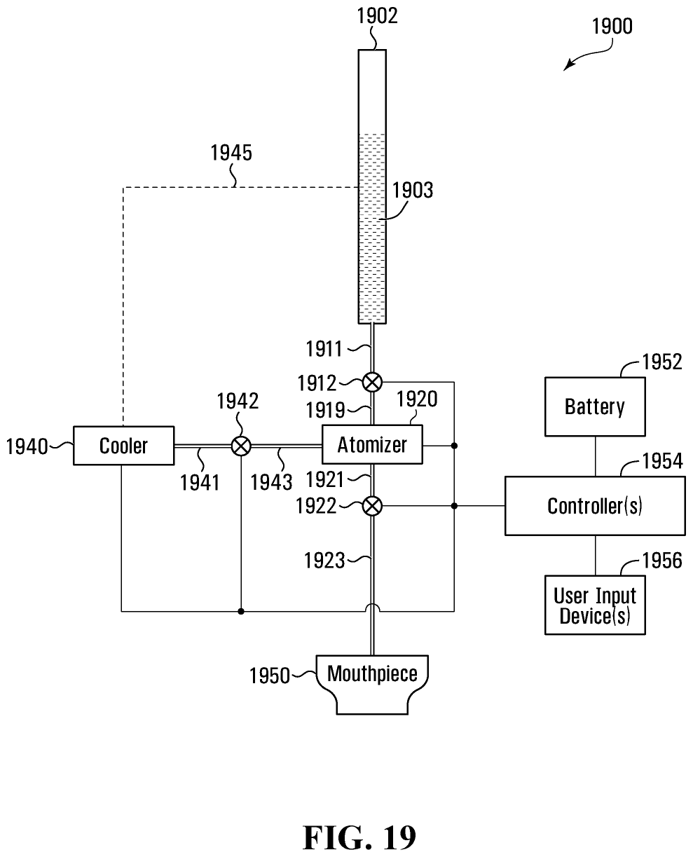

The embodiments described above largely relate to coolers that are implemented downstream of an atomizer in the direction of air and vapor flow. Coolers that are implemented upstream of an atomizer in the direction of air flow are also contemplated. FIG. 19 is a block diagram illustrating an example vaporization device 1900. The vaporization device 1900 includes a chamber 1902 to store a vaporization substance 1903.

The chamber 1902 may be similar to any of the chambers described elsewhere herein. In some embodiments, the chamber 1902 includes an engagement structure to engage with a complementary engagement structure of the example device 1900. These engagement structures may limit the example device 1900 to certain types of chambers.

The chamber 1902 may be recloseable or non-recloseable. Examples of releasable engagements for recloseable chambers and non-releasable engagements or non-recloseable chambers are provided elsewhere herein.

An atomizer 1920 is in fluid communication with the chamber 1902 through channels 1911, 1919 and a regulator in the form of a valve 1912 in the example shown. The valve 1912 controls movement of the vaporization substance 1903 from the chamber 1902. Other examples of regulators are disclosed elsewhere herein.

The valve 1912 is in fluid communication with the atomizer 1920 through the channel 1919. In some embodiments, the valve 1912 is integrated with the atomizer 1920 in a single component. The valve 1912 controls the movement of the vaporization substance 1903 to the atomizer 1920, which generates a vapor by heating the vaporization substance. The atomizer 1920 includes a heater to heat the vaporization substance, and could be implemented as described elsewhere herein by way of example.

The vapor produced by the atomizer 1920 is fed into a channel 1921. The channel 1921 is in fluid communication with the atomizer 1920, to carry the vapor away from the atomizer. A vapor valve 1922, which is an example of a vapor regulator, is provided to control a flow of the vapor from the atomizer 1920.

A mouthpiece 1950 is in fluid communication with the atomizer 1920, through the channels 1921, 1923 and a vapor valve 1922 therebetween. The vapor valve 1922 controls the flow of vapor to the mouthpiece 1950.

The vaporization device 1900 further includes channels 1941, 1943. The channels 1941, 1943 provide an air intake channel, in fluid communication with the atomizer 1920, to carry air to the atomizer 1920. The channels 1941, 1943 are also in fluid communication with a source of air, an example of which is an air inlet to draw air from the outside environment.

A cooler 1940 is provided to cool air entering the atomizer 1920. The cool air may be introduced to the channel 1941 by or through the cooler 1940, or the cooler may cool air that is already in the channel 1941. The channels 1941, 1943 then carry the cool air to the atomizer 1920. When cooled air is mixed with vapor generated in the atomizer 1920, the vapor is cooled. An air valve 1942 is provided between the channels 1941, 1943 to control the flow of cooled air to the atomizer 1920, and thereby control cooling of the vapor. The air valve 1942 is integrated with the cooler 1940 in some implementations.

The cooler 1940 may be similar to any cooler disclosed herein, such as the cooler 500 of FIG. 5, for example. In some implementations, the cooler 1940 is in fluid communication with the channel 1941. For example, at least a portion of the cooler 1940 may be located at a position inside the channel 1941. The cooler 1020 of FIGS. 10 and 11 is an example of a cooler that is disposed at least partially inside of a channel. At least a portion of the cooler 1940 may also or instead be in contact with and/or engaged with an outside of the channel 1941 to indirectly cool the air. For example, the cooler 1940 may be similar to the cooler 1220 of FIGS. 12 to 14.

In some implementations, the cooler 1940 includes a source of air. By way of example, a compressed air tank may provide a source of air. When air is released from the compressed air tank, the air expands and cools. Thus, cool air could be provided by a compressed air tank without the use of further cooling elements. However, one or more further cooling elements are provided to further cool air from a compressed air tank in some implementations. An air inlet is another example of a source of air that may be included in the cooler 1940.

The cooler 1940 may include one or more cooling elements, including a heat sink that has been refrigerated prior to use, and/or a thermoelectric cooling element. In some implementations, the cooler 1940 includes a heat exchanger to transfer heat to the chamber 1902. The optional transfer of heat to the chamber 1902 is illustrated in FIG. 19 using a dashed line at 1945.

The valve 1912, the atomizer 1920, the vapor valve 1922, the air valve 1942, and the cooler 1940 are controlled by one or more controllers 1954. A power source such as a battery 1952 and one or more user input devices 1956 are coupled to the controller(s) 1954. The controller(s) 1954, the user input device(s) 1956, and the battery 1952 could be similar to components disclosed in FIG. 6, for example. Examples of power and/or control connections that could be implemented in the vaporization device 1900 are also provided elsewhere herein. In some implementations, the cooler 1940 includes additional controller(s) and/or user input device(s) for controlling the cooler. The cooler 1940 may be controlled, by the controller(s) 1954 or another controller in the cooler, responsive to input from a user received by one or more of the user input device(s) 1956 for example.

In some implementations, one or more sensors may be disposed downstream of the atomizer 1920, in the channel 1923 and/or in the mouthpiece 1950 for example, to measure a temperature of the vapor. Sensors could also or instead be implemented in either or both of the channels 1941, 1943 to measure a temperature of air entering the atomizer 1920. The cooler 1940 may then be controlled, by the controller(s) 1954 or another controller in the cooler, responsive to one or more measurements of air/vapor temperature by one or more sensors.

The position of the cooler 1940 with respect to the atomizer 1920 is implementation specific. In some cases, the position of the cooler 1940 is based on the expected temperature rise of cool air in the channels 1941, 1943. The cooler 1940 may be positioned proximate to the atomizer 1920 to limit the length of channel that cooled air traverses before mixing with the vapor, for example.

A specific example of a vaporization device 1900 is shown in FIG. 1900. Other embodiments are also contemplated. For example, multiple chambers to store respective vaporization substances may be provided. A chamber may be in fluid communication with a respective atomizer, multiple chambers may supply their respective vaporization substances to the same atomizer, and/or one or more chambers may supply their vaporization substance(s) to a channel or other component and not directly to an atomizer. Multiple channels, in fluid communication with different atomizers, chambers, coolers or air intakes for example, may be provided.

Any or all of the valve 1912, the vapor valve 1922, and the air valve 1942 may be excluded from other vaporization devices. Valves may also or instead be provided in different channels.

More than one cooler 1940 may be provided in some embodiments. The additional cooler(s) may be implemented in fluid communication with the atomizer 1920, upstream and/or downstream of the atomizer in a direction of vapor and air flow.

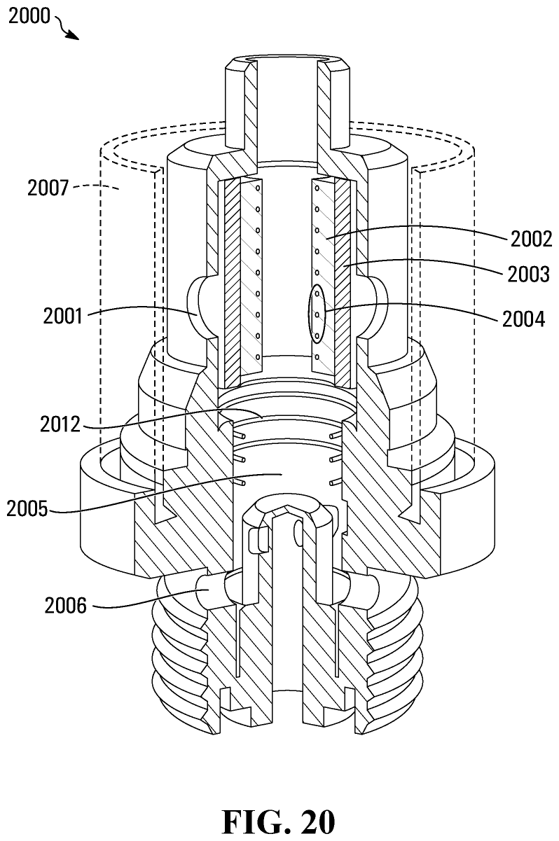

FIG. 20 is a diagram illustrating internal structure of an example vaporization device tank 2000 with a cooler 2012. The example vape tank 2000 is shown with a section removed so that internals of the vape tank can be seen. The vape tank 2000 can be implemented in a vaporization device, non-limiting examples of which are provided elsewhere herein.

The vape tank 2000 includes a chamber 2007 to store a vaporization substance. Example implementations of chambers are provided elsewhere herein. An inlet 2001 fluidly connects the chamber 2007 to a wick 2003. The wick 2003 is adjacent to a ceramic core 2002 with a heating element 2004. The wick 2003 and the ceramic core 2002 provide an atomizer to generate vapor from the vaporization substance.

The vape tank 2000 further includes an air inlet 2006. The air inlet 2006 forms part of an air intake channel 2005, in fluid communication with the ceramic core 2002, to carry air to the ceramic core.

Illustrative examples of these components of the example vape tank 2000 are provided at least above, with reference to FIG. 4.

A cooler 2012 is located at a position inside the air intake channel 2005, and cools the air in the air intake channel. The cool air then mixes with vapor generated by the ceramic core 2002, and cools the vapor. FIG. 20 is intended to illustrate the cooler 2012 as a multi-turn coil, which is an example of a surface area increasing structure to increase a surface area for heat transfer. The number of turns in the coil is provided by way of example. More or fewer turns, other forms of a surface area increasing structure, and/or other types of cooler or cooling elements may be implemented in other embodiments.

In some implementations, the cooler 2012 includes a heat sink that is at a temperature below the temperature of air entering the air inlet 2006. For example, the heat sink could be a removable heat sink element that is refrigerated before use. The heat sink could also or instead include a cooled fluid that is circulated through the cooler 2012, where the coils of the cooler 2012 could provide a channel to carry the fluid. An active cooling element such as a thermoelectric cooler, for example, could be used to cool the fluid.

The cooler 2012 may also or instead include one or more thermoelectric cooling elements, in the coils as shown or in another form. The hot side of a thermoelectric cooling element may be in contact with a thermal conductor, or another form of heat exchanger, to conduct heat away from the air intake channel 2005. The cold side of such a thermoelectric cooling element may directly cool the air in the air intake channel 2005. Power and/or control connections for a thermoelectric cooling element, and/or other active cooler(s) or cooling element(s), may be implemented on the walls of the air intake channel 2005 or otherwise inside the air intake channel, for example.

The cooler 2012 may be positioned in the air intake channel 2005 to avoid or reduce interaction or interference with the ceramic core 2002, for example. An axial and/or radial separation distance between the cooler 2012 and the ceramic core 2002 may be selected to reduce cooling of the ceramic core by the cooler, to reduce heating of the cooler by the ceramic core, and/or based on any of various other factors.

The cooler 2012 is one example of a cooler to cool air in an air intake channel. Other examples are also contemplated. In some implementations, a cooler cools the outer walls of an air intake channel to indirectly cool the air inside the air intake channel. Such a cooler may or may not be in fluid communication with the air intake channel. Instead of or in addition to coils, a cooler may include one or more cooling elements in the form of a sleeve around the inside or outside of an air intake channel, for example.

Other variations of vaporization apparatus such as vaporization devices, coolers, cooling elements, and/or other components may be or become apparent to those skilled in the art.

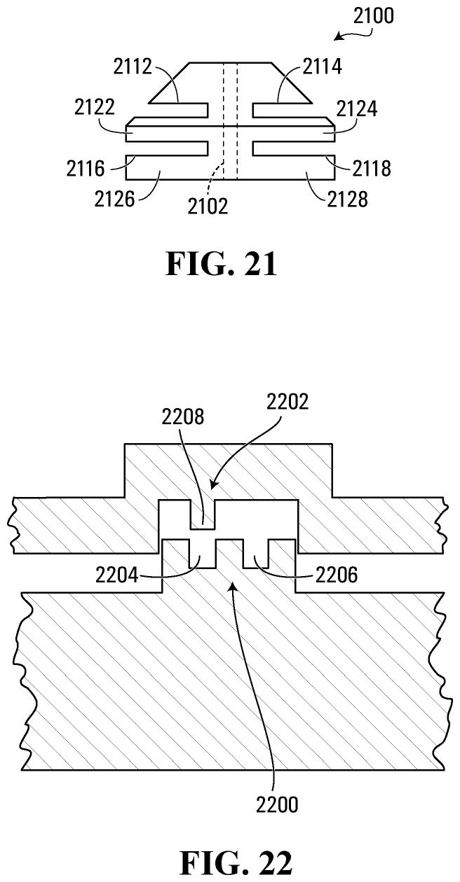

As an example, FIG. 21 illustrates a plan view of a cap according to another embodiment. The cap 2100 includes a central channel 2102 to enable fluid flow through the cap. The cap 2100 is also tapered at its top in the example shown, and may provide a mouthpiece through which a user may inhale vapor. Although not explicitly shown, the cap 2100 may engage a chamber and/or a stem of a vaporization device, for example. Cap features, materials, and variations that are disclosed elsewhere herein may also or instead be embodied by the cap 2100.

Notches or grooves 2112, 2114, 2116, 2118 define fins 2122, 2124, 2126, 2128 in the cap 2100, which may extend partially or entirely around the perimeter of the cap 2100. In a generally cylindrical cap, for example, annular notches or grooves 2112/2114, 2116/2118 define annular fins 2122/2124, 2126/2128. In another embodiment, notches or grooves also or instead extend axially, and/or in one or more other directions, to define or further define fins on a cap or mouthpiece. One or more parameters or characteristics such as the number, size(s), and/or orientation(s) of cap or mouthpiece cooling fins may be determined based on any of various factors, including the expected or measured temperature examples provided elsewhere herein.

In any case, surface area increasing structures such as the notches or grooves 2112, 2114, 2116, 2118 and the fins 2122, 2124, 2126, 2128 may aid in diffusing heat from a fluid stream as it flows through the channel 2102 in a cap or mouthpiece. The embodiment shown in FIG. 21 is illustrative of structural features of a cap or mouthpiece to cool a fluid that flows through a channel of a vaporization device or a channel thereof.

In a variation of the embodiment shown in FIG. 21, the notches or grooves 2112, 2114, 2116, 2118 and/or the fins 2122, 2124, 2126, 2128 are made from, are coated with, or otherwise include or carry one or more thermally conductive materials such as metals. This may improve transfer of heat from the channel 2102 to ambient air. The notches or grooves 2112, 2114, 2116, 2118 and/or the fins 2122, 2124, 2126, 2128 may also or instead be made from, be coated with, or otherwise include one or more heat sinks, to potentially increase heat absorption capacity of a cap or cover and thereby improve cooling of fluid in the channel 2102.

Although the fins in FIG. 21 are exterior fins, a cap or mouthpiece may include one or more interior structures to aid in fluid cooling. Cooling features that are described elsewhere herein, for a vaporization device stem and/or other parts of a vaporization device channel for example, may also be applied to a cap or mouthpiece.

Several embodiments herein reference chamber engagement structures. FIG. 22 is a cross-sectional and partially exploded view of an example of engagement structures in a vaporization device. FIG. 22 illustrates an engagement structure 2200 and a complementary engagement structure 2202. Engagement structures may be used with replaceable or reconfigurable secondary chambers in a vaporization device. These engagement structures may be useful for restricting a vaporization device to a particular model or type of chamber or cartridge. Engagement structures may also or instead be useful as an assembly aid, to ensure that chambers or cartridges are assembled or installed properly. Further, the engagement structure for a chamber or cartridge may include or provide an indicator of the vaporization substance stored in the chamber or cartridge, and/or a type of the chamber or cartridge. A vaporization device may then read this indicator to determine the type of vaporization substance, chamber, and/or cartridge. For example, some chambers or cartridges may include one or more active coolers, and a vaporization device may adapt power supply and/or control to a chamber or cartridge according to chamber or cartridge type.

In the example of FIG. 22, the presence of the protrusion 2208 aligned with the notch 2204 and the lack of a protrusion aligned with the notch 2206 may provide information regarding an installed chamber. This information may include the type of vaporization substance stored by a chamber, which may be used by a controller, in a base of a vaporization device, for example, to control the voltage, current, and/or power supplied to an atomizer and/or an active cooler. One or more regulators may also or instead be controlled based on the type of vaporization substance stored by a chamber or cartridge.

This is just one example of how fluid cooling control may be automated in some embodiments.

Various embodiments are described herein as illustrative examples. More generally, some embodiments may be summarized as relating to a vaporization apparatus that includes: an atomizer to generate vapor from a vaporization substance by heating the vaporization substance; a channel, in fluid communication with the atomizer, to enable fluid flow through the vaporization apparatus; and a cooler to cool the fluid. The channel may include, for example, a vaporization device stem, one or more intake channels, and/or a channel through a mouthpiece.

A cooler may be thermally coupled to the channel, to indirectly cool the fluid that is flowing in the channel by cooling at least part of the channel. A cooler may be in fluid communication with the channel, to directly cool the fluid as the fluid flows through, past, and/or around the cooler or one or more cooling elements of the cooler. At least a portion of the cooler may be located inside the channel, for example.

The channel of a vaporization apparatus may include or be in fluid communication with an air intake channel, that is at least in fluid communication with the atomizer, to carry air to the atomizer. In some embodiments, the cooler is thermally coupled to the air intake channel to cool the air indirectly by cooling the air intake channel. The cooler may be in fluid communication with the air intake channel, to cool the air directly. For example, at least a portion of the cooler may be located inside the air intake channel.

A cooling air intake channel may also or instead be provided, in fluid communication with the channel, to admit cooling air into the channel to mix with the vapor. Examples of vapor cooling by mixing with air are described at least above. A vaporization apparatus may include a regulator to control a flow of the cooling air through a cooling air intake channel. Such a regulator may be part of a cooler or a separate component.

A cooler may be or include a passive cooling element such as a thermally conductive material, illustratively copper, to transfer heat away from the fluid. A cooler that is or includes an active cooling element is also possible, and a thermoelectric cooling element is an example.

In the case of an active cooler, a vaporization apparatus may also include a power source to power the cooler. The power source may, but need not be, dedicated to powering only the cooler. The power source may be further arranged, by connections in the vaporization apparatus for example, to power the atomizer. Other components may also or instead be powered by the same power source.

One or more sensors may be provided, for example, to measure a temperature of the fluid. A controller may be in communication with or otherwise coupled to the sensor(s), to control an active cooler, and/or other components such as one or more regulators to regulate flow of one or more cooling media, responsive to one or more temperature measurements by the sensor(s).

One or more user input devices may be provided to receive input from a user, and a controller may be coupled to the user input device to control the cooler and/or other components responsive to the input from the user. Control may be based on multiple inputs or parameters, such as one or more sensor measurements and one or more user inputs.

Whether active or passive, a cooler may include a surface area increasing structure to increase a surface area for heat transfer. Examples include one or more fins and a coil with one or more turns.

A cooler may be or include a heat sink, with one or more substances or materials such as air, a liquid, and/or a phase change material to absorb heat.

One or more heat exchangers may be provided, to transfer heat to such a heat sink for example. Other applications of one or more heat exchangers are possible, including to also or instead transfer heat to the atomizer, transfer heat to a chamber in which vaporization substance is stored prior to vaporization, and/or transfer heat away from the channel.

In some embodiments, a cooler is or includes one or more removable cooling elements, which may be coupled to the vaporization apparatus magnetically and/or by another type of releasable coupling.

A cooler, or at least a portion thereof, may be provided on or in a mouthpiece that enables inhalation of vapor by a user through the channel. Examples include at least those shown in FIGS. 7, 8, 16, 17, and 21.

Another example of a cooling features that may be implemented in conjunction with a mouthpiece is shown in FIG. 18, in which a hose or pipe such as 1832, 1842, in fluid communication with a vaporization apparatus channel and a mouthpiece such as 1834, 1844, in effect lengthens the channel and/or otherwise enables cooling of vapor before inhalation. One or more cooling elements to cool fluid during flow through such further channel(s) provided by a hose or pipe 1832, 1842, may provide or improve cooling of vapor before inhalation.

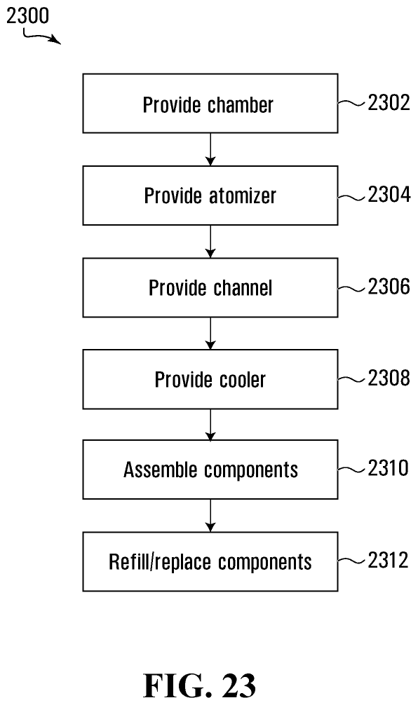

Embodiments described above relate primarily to vaporization apparatus such as vaporization devices. Other embodiments, including methods, are also contemplated. FIG. 23, for example, is a flow diagram illustrating a method according to an embodiment.

The example method 2300 includes several operations relating to providing components of a vaporization apparatus. The set of components provided in any embodiment depends on the nature or type of the vaporization apparatus. For example, components for a complete vaporization device, or components for only part of a vaporization device for subsequent assembly or combination with other components, may be provided. For example, although the example method 2300 includes an operation 2302 of providing a chamber to store a vaporization substance, in some embodiments a method may include an operation 2304 of providing an atomizer to generate vapor from the vaporization substance by heating the vaporization substance, an operation 2306 of providing an insulated channel to carry the vapor away from the atomizer and/or otherwise enable fluid flow through the vaporization apparatus, and an operation 2308 of providing a cooler to cool the vapor or fluid, without necessarily also providing a chamber.

These operations 2302, 2304, 2306, 2308 are shown separately for illustrative purposes, but need not be separate operations in all embodiments. For example, a vaporization device or cartridge may include a chamber, an atomizer, a channel such as a stem, and a cooler. A vaporization device, or components thereof, may potentially be provided and/or purchased separately from chambers, for example. Some chambers may be provided with a vaporization device, while others may be sold separately. Therefore, the operations 2302, 2304, 2306, 2308 need not necessarily be separate operations, and any two or more of these operations may be performed together.

A chamber, atomizer, channel, and/or cooler may be provided at 2302, 2304, 2306, 2308 by actually manufacturing these components. Any of these components, and/or other components, may instead be provided by purchasing or otherwise acquiring the components from one or more suppliers.

At least some components or parts thereof may be provided in different ways. Different cartridge parts, such as chambers, bases, caps, atomizers, stems, and/or coolers, for example, may be provided by manufacturing one or more parts and purchasing one or more other parts, or by purchasing different parts from different suppliers.

In some embodiments, components such as the atomizer provided at 2304, the channel provided at 2306, the cooler provided at 2308, and possibly the chamber provided at 2302, are provided in the form of a pre-assembled vaporization device. In other embodiments, components are not necessarily assembled. FIG. 23 therefore also illustrates an operation 2308 of assembling components. This may involve, for example, arranging the atomizer in fluid communication with the chamber and/or the channel, such as by installing the atomizer, the channel, and/or the chamber in a vaporization device or cartridge.

Assembling components at 2310 may also or instead involve installing or arranging the cooler in any of various ways. For example, providing the cooler at 2308 may involve providing, as the cooler, a cooler that is to be thermally coupled to the channel, in which case the assembling at 2310 may involve arranging or installing the cooler by thermally coupling the cooler to the channel. As another example, providing the cooler at 2308 may involve providing, as the cooler, a cooler that is to be in fluid communication with the channel, and the assembling at 2310 may then involve arranging or installing the cooler by placing it in fluid communication with the channel. In some embodiments, providing the cooler at 2308 involves providing, as the cooler, a cooler that is to be at least partially located inside the channel, and the assembling at 2310 may involve locating, positioning, or otherwise installing the cooler at least partially inside the channel.

Other assembly operations may also or instead be performed, depending on the components or elements that are provided and how those components are to interact with each other. For example, apparatus or device embodiments disclosed herein involve various components or elements that are in fluid communication with each other, thermally coupled to each other, and/or otherwise connected or coupled together, and one or more operations to implement such connections or coupling of components or elements together may be performed at 2310.

A method may include other operations as well. FIG. 23 includes an example at 2312, in which one or more components, such as a chamber, may be refilled or replaced.

The example method 2300 is illustrative of one embodiment. Examples of various ways to perform the illustrated operations, additional operations that may be performed in some embodiments, and/or operations that could be omitted in some embodiments, could be inferred or apparent from the description and drawings, for example. Further variations may be or become apparent.

For example, the channel provided 2306 may be or include an air intake channel to carry air to the atomizer. Embodiments in which the cooler provided at 2308 is to be thermally coupled to, in fluid communication with, or at least partially located inside such an air intake channel are possible, and are described in further detail elsewhere herein.

In some embodiments, the cooler provided at 2308 includes a cooling air intake channel to admit cooling air into the channel to mix with the vapor from the atomizer. A regulator to control a flow of the cooling air through the cooling air intake channel may be provided in some embodiments. This type of regulator is illustrative of components that may be provided, as part of another component or separately, in some embodiments.

Other features disclosed herein may be applied to method embodiments, even if disclosed in different embodiments such as apparatus embodiments. For example, providing the cooler at 2308 may involve providing a cooler that is or includes one or more passive cooling elements such as copper and/or one or more other thermally conductive materials, and/or providing a cooler that is or includes one or more active cooling elements such as a thermoelectric cooling element. A cooler that also or instead includes one or more surface area increasing structures to increase surface area for heat transfer, one or more heat sinks, and/or one or more heat exchangers may be provided at 2308. Providing a cooler at 2308 may also or instead involve providing a cooler that includes one or more removable cooling elements that can be coupled to a vaporization apparatus by a releasable coupling, such as magnetically. Examples of surface area increasing structures, heat sinks, heat exchangers, removable cooling elements, and other releasable couplings are provided elsewhere herein.

In the case of providing an active cooler that includes one or more active cooling elements, a method may involve providing such components as a power source to power the cooler or at least the active cooling element(s), one or more sensors to measure a temperature of the fluid in the channel, one or more user input devices to receive input from a user, and/or a controller. A power source may be provided and connected or otherwise arranged or installed to power only the active cooling element(s), or to power other components such as the atomizer as well. A controller may be provided, and coupled to the sensor(s) and/or to the user input device(s), to control the cooler responsive to one or more temperature measurements by the sensor(s) and/or responsive to one or more inputs received from the user through the user input device(s).

A mouthpiece to enable inhalation of vapor by a user through the channel is another example of a component that may be provided. The mouthpiece may include at least part of the cooler in some embodiments.

Another example of a cooling features that may be implemented in conjunction with a mouthpiece is shown in FIG. 18, in which one or more further channels such as a hose or pipe such as 1832, 1842, are to be in fluid communication with a vaporization apparatus channel and a mouthpiece such as 1834, 1844. A longer fluid flow path provided by such further channel(s), and/or one or more cooling elements to cool fluid during flow through the further channel(s), may provide or improve cooling of vapor before inhalation.

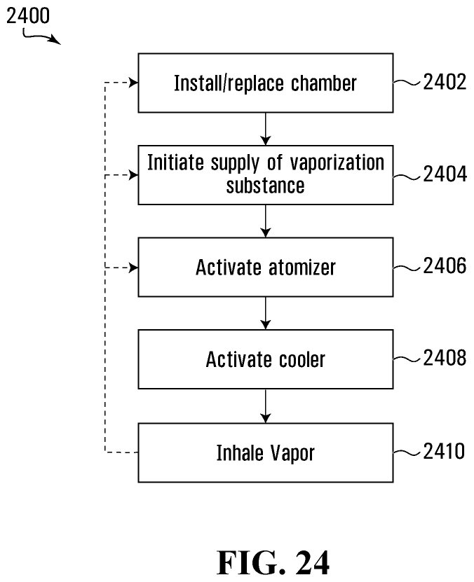

User methods are also contemplated. FIG. 24 is a flow diagram illustrating a method according to another embodiment.

The example method 2400 involves an optional operation 2402 of installing or replacing a chamber. A user need not necessarily install or replace a chamber every time a vaporization substance is to be vaporized. The example method 2400 also involves an operation 2404 of initiating supply of a vaporization substance from the chamber to an atomizer, an operation 2406 of activating the atomizer, and an operation 2408 of activating a cooler. These operations may involve operating one or more input devices such as a control button or switch or even just inhaling on a mouthpiece. The operations at 2404, 2406, 2408 are shown separately in FIG. 24 solely for illustrative purposes, and need not necessarily be separate operations.

Similarly, inhaling vapor through a channel is represented separately at 2410, but in some embodiments inhaling on a mouthpiece initiates vaporization substance flow, vaporization, and cooling. The vapor that a user inhales at 2410 is cooled vapor according to embodiments disclosed herein.

The dashed arrows in FIG. 24 illustrate that multiple doses of a vaporization substance may be vaporized, and that a vaporization substance may be changed by installing or replacing a chamber at 2402.

The example method 2400 is an illustrative and non-limiting examples. Various ways to perform the illustrated operations, additional operations that may be performed in some embodiments, and/or operations that may be omitted in some embodiments, may be inferred or apparent from the description and drawing or otherwise be or become apparent.

Illustrative embodiments have been described with reference to specific features and examples, various modifications and combinations can be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although embodiments and potential advantages have been described by way of example in detail, various changes, substitutions and alterations can be made herein without departing from the invention as defined by the appended claims. Moreover, the scope of the present application is not intended to be limited to the particular embodiments of any process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A vaporization apparatus comprising:
an atomizer to generate vapor from a vaporization substance by heating the vaporization substance;
a channel, in fluid communication with the atomizer, to enable fluid flow through the vaporization apparatus; and
a cooler to cool the fluid, wherein the cooler comprises a cooling air intake channel, in fluid communication with the channel, to admit cooling air into the channel to mix with the vapor.

2-7. (canceled)

8. The vaporization apparatus of claim 1, wherein the channel comprises an air intake channel in fluid communication with the atomizer to carry air to the atomizer, wherein at least a portion of the cooler is located inside the air intake channel.

9. (canceled)

10. The vaporization apparatus of claim 1, further comprising:
a regulator to control a flow of the cooling air through the cooling air intake channel.

11. The vaporization apparatus of claim 1, wherein the cooler comprises a passive cooling element.

12-13. (canceled)

14. The vaporization apparatus of claim 1, wherein the cooler comprises an active cooling element.

15. The vaporization apparatus of claim 14, wherein the active cooling element comprises a thermoelectric cooling element.

16-23. (canceled)

24. A vaporization apparatus comprising:
an atomizer to generate vapor from a vaporization substance by heating the vaporization substance;
a channel, in fluid communication with the atomizer, to enable fluid flow through the vaporization apparatus; and
a cooler to cool the fluid, wherein the cooler comprises a heat exchanger to transfer heat to the atomizer or to a chamber to store the vaporization substance.

25-28. (canceled)

29. The vaporization apparatus of claim 1, wherein the cooler comprises a removable cooling element, wherein the removable cooling element is coupled to the vaporization apparatus by a releasable coupling, wherein the removable cooling element is coupled to the vaporization apparatus magnetically.

30. The vaporization apparatus of claim 14, further comprising:
a power source to power the cooler.

31. The vaporization apparatus of claim 30, wherein the power source is further arranged to power the atomizer.

32. The vaporization apparatus of claim 14, further comprising:
a sensor to measure a temperature of the fluid; and
a controller, coupled to the sensor, to control the cooler responsive to a temperature measurement by the sensor.

33. The vaporization apparatus of claim 14, further comprising:
a user input device to receive input from a user; and
a controller, coupled to the user input device, to control the cooler responsive to the input from the user.

34-35. (canceled)

36. A method of use of the vaporization apparatus of claim 1, the method comprising:
initiating vaporization of the vaporization substance to produce the vapor; and
inhaling the vapor through the channel.

37. The method of claim 36, further comprising:
initiating cooling of the vapor by the cooler prior to inhaling the vapor.

38. A method comprising:
providing an atomizer for a vaporization apparatus to generate vapor from a vaporization substance by heating the vaporization substance;
providing a channel to enable fluid flow through the vaporization apparatus; and
providing a cooler to cool the fluid,
wherein providing the cooler comprises providing, as the cooler, a cooler that comprises a cooling air intake channel to admit cooling air into the channel to mix with the vapor.

39-44. (canceled)

45. The method of claim 38, wherein the channel comprises an air intake channel to carry air to the atomizer, wherein providing the cooler comprises providing, as the cooler, a cooler that is to be at least partially located inside the air intake channel.

46. (canceled)

47. The method of claim 38, further comprising:
providing a regulator to control a flow of the cooling air through the cooling air intake channel.

48. The method of claim 38, wherein providing the cooler comprises providing, as the cooler, a cooler that comprises a passive cooling element.

49-50. (canceled)

51. The method of claim 38, wherein providing the cooler comprises providing, as the cooler, a cooler that comprises an active cooling element.

52. The method of claim 51, wherein the active cooling element comprises a thermoelectric cooling element.

53-60. (canceled)

61. The method of claim 38, wherein the cooler comprises a heat exchanger to transfer heat to the atomizer or to a chamber to store the vaporization substance.

62-65. (canceled)

66. The method of claim 38, wherein the cooler comprises a removable cooling element, wherein the removable cooling element is couplable to the vaporization apparatus by a releasable coupling, wherein the removable cooling element is couplable the vaporization apparatus magnetically.

67. The method of claim 51, further comprising:
providing a power source to power the cooler.

68. The method of claim 67, wherein providing the power source comprises providing the power source to further power the atomizer.

69. The method of claim 51, further comprising:
providing a sensor to measure a temperature of the fluid; and
providing a controller to control the cooler responsive to a temperature measurement by the sensor.

70. The method of claim 51; further comprising:
providing a user input device to receive input from a user; and
providing a controller to control the cooler responsive to the input from the user.

71-72. (canceled)
